SEQUENCE LISTING (10 sequences)

SEQ ID NO: 1 - protein, 190 aa, Cowpea chlorotic mottle virus
MSTVGTGKLTRAQRRAAARKNKRNTRVVQPVIVEPIASGQGKAIKAWTGYSVSKWTASCAAAEAKVTSAITISLPNELSSERNKQLKVGRVLLWLGLLPSVSGTVKSCVTETQTTAAASFQVALAVADNSKDVVAAMYPEAFKGITLEQLTADLTIYLYSSAALTEGDVIVHLEVEHVRPTFDDSFTPVY

SEQ ID NO: 2 - protein, 374 aa, Cowpea mosaic virus
MEQNLFALSLDDTSSVRGSLLDTKFAQTRVLLSKAMAGGDVLLDEYLYDVVNGQDFRATVAFLRTHVITGKIKVTATTNISDNSGCCLMLAINSGVRGKYSTDVYTICSQDSMTWNPGCKKNFSFTFNPNPCGDSWSAEMISRSRVRMTVICVSGWTLSPTTDVIAKLDWSIVNEKCEPTIYHLADCQNWLPLNRWMGKLTFPQGVTSEVRRMPLSIGGGAGATQAFLANMPNSWISMWRYFRGELHFEVTKMSSPYIKATVTFLIAFGNLSDAFGFYESFPHRIVQFAEVEEKCTLVFSQQEFVTAWSTQVNPRTTLEADGCPYLYAIIHDSTTGTISGDFNLGVKLVGIKDFCGIGSNPGIDGSRLLGAIAQ

SEQ ID NO: 3 - protein, 189 aa, Cowpea mosaic virus
GPVCAEASDVYSPCMIASTPPAPFSDVTAVTFDLINGKITPVGDDNWNTHIYNPPIMNVLRTAAWKSGTIHVQLNVRGAGVKRADWDGQVFVYLRQSMNPESYDARTFVISQPGSAMLNFSFDIIGPNSGFEFAESPWANQTTWYLECVATNPRQIQQFEVNMRFDPNFRVAGNILMPPFPLSTETPPL

SEQ ID NO: 4 - protein, 188 aa, Physalis mottle virus
MDSSEVVKVKQASIPAPGSILSQPNTEQSPAIVLPFQFEATTFGTAETAAQVSLQTADPITKLTAPYRHAQIVECKAILTPTDLAVSNPLTVYLAWVPANSPATPTQILRVYGGQSFVLGGAISAAKTIEVPLNLDSVNRMLKDSVTYTDTPKLLAYSRAPTNPSKIPTASIQISGRIRLSKPMLIAN

SEQ ID NO: 5 - protein, 268 aa, Sesbania mosaic virus
MAKRLSKQQLAKAIANTLETPPQPKAGRRRNRRRQRSAVQQLQPTQAGISMAPSAQGAMVRIRNPAVSSSRGGITVLTHSELSAEIGVTDSIVVSSELVMPYTVGTWLRGVAANWSKYSWLSVRYTYIPSCPSSTAGSIHMGFQYDMADTVPVSVNQLSNLRGYVSGQVWSGSAGLCFINGTRCSDTSTAISTTLDVSKLGKKWYPYKTSADYATAVGVDVNIATPLVPARLVIALLDGSSSTAVAAGRIYCTYTIQMIEPTASALNN

SEQ ID NO: 6 - protein, 190 aa, Cowpea chlorotic mottle virus; identical to SEQ ID NO: 1

SEQ ID NO: 7 - protein, 374 aa, Cowpea mosaic virus; identical to SEQ ID NO: 2

SEQ ID NO: 8 - protein, 189 aa, Cowpea mosaic virus; identical to SEQ ID NO: 3

SEQ ID NO: 9 - protein, 188 aa, Physalis mottle virus; identical to SEQ ID NO: 4

SEQ ID NO: 10 - protein, 268 aa, Sesbania mosaic virus; identical to SEQ ID NO: 5

(19) United States
(12) Patent Application Publication
(10) Pub. No.: US 2025/0017991 A1
(43) Pub. Date: Jan. 16, 2025

(54) VLP IMMUNOTHERAPY COMBINED WITH ABLATION

(71) Applicant: The Regents of the University of California, Oakland, CA (US)

(72) Inventors: Nicole F. Steinmetz, La Jolla, CA (US); Isabel Newton, La Jolla, CA (US); Nicholas Webster, La Jolla, CA (US); Tyler Mandt, La Jolla, CA (US); Hui Cai, La Jolla, CA (US); Christine Boone, La Jolla, CA (US); Mansur Ghani, La Jolla, CA (US)

(21) Appl. No.: 18/708,930

(22) PCT Filed: Nov. 9, 2022
(86) PCT No.: PCT/US2022/049450

§ 371 Date: May 9, 2024

Related U.S. Application Data
(60) Provisional application No. 63/278,457, filed on Nov. 11, 2021.

(51) Int. Cl. A61K 35/768; A61B 18/02; A61P 35/00; C07K 16/28; C12N 7/00

(52) U.S. Cl. CPC A61K 35/768; A61B 18/02; A61P 35/00; C07K 16/2818; C12N 7/00; C12N 2770/18023; C12N 2770/18032

(57) ABSTRACT

Provided herein is a novel, multimodal in situ vaccination approach to treat cancer by combining ablation with administration of the naturally occurring plant virus or virus-like particle (VLP) nanoparticles such as cowpea mosaic virus (CPMV), a plant virus-based immunoadjuvant.

### CROSS-REFERENCE TO RELATED APPLICATION

This application claims priority under 35 U.S.C. § 119 (e) to U.S. Provisional Application No.: 63/277,737, filed Nov. 10, 2021 and U.S. Provisional Application No. 63/278,457, filed Nov. 11, 2021, the contents of each of which is incorporated herein by reference in their entirety.

### STATEMENT OF GOVERNMENT SUPPORT

This invention was made with government support under Grant No. W81XWH2010742 awarded by the United States Department of Defense (DoD), and Grant Nos. EB005970, CA224605, and CA218292 awarded by the National Institutes of Health. The government has certain rights in the invention.

### BACKGROUND

The following discussion of the background is merely provided to aid the reader in the understanding the disclosure and is not admitted to describe or constitute prior art to the present disclosure. Throughout and within this disclosure, various patent and technical publications are referenced by an identifying citation or an Arabic number, the full bibliographic citation for which can be found immediately preceding the claims. These disclosures are incorporated herein to more fully describe the state of the art to which this disclosure pertains.

Hepatocellular carcinoma (HCC) is the 4^{th} cause of cancer-related death worldwide. The US incidence has almost tripled in 30 years. HCC evades immune detection and capitalizes on the liver's immunosuppressive microenvironment, properties that immunotherapy aims to reverse. Checkpoint inhibitors (CPI) targeting programmed cell death protein 1 (PD-1) and its ligand PD-L1 are treatments for advanced HCC. However, phase 3 clinical trials of CPIs nivolumab or pembrolizumab as single agents versus sorafenib or versus best supportive care for HCC failed to improve survival. Combination CPI atezolizumab plus VEGF inhibitor bevacizumab improved survival versus sorafenib, earning it FDA approval as first-line therapy in unresectable HCC. Due to the immunosuppressive tumor microenvironment (TME) in HCC, combining immunotherapy with liver-directed therapies may constitute a robust in situ vaccination (ISV) approach. Thus, a need exists in the art to treat HCC and related disorders. This disclosure satisfies this need and provides related advantages, as well.

### SUMMARY OF THE DISCLOSURE

Disclosed herein is a multi-modal in situ vaccination approach to treat local and metastatic cancer by combining ablation such as cryoablation to kill tumor cells and release tumor-associated antigens with a plant virus-based nanotechnology with demonstrated immunostimulatory efficacy. Specifically, VLP nanoparticles, e.g., from the cowpea mosaic virus (CPMV) are delivered into the tumor at the time of cryoablation, as CPMV is a highly potent adjuvant; when administered intratumorally, CPMV-primed immunostimulation leads to recruitment and activation of innate immune cells to process tumor-associated antigens released by the ablation. Although in one aspect the VLPs are delivered intratumorally, they also can be delivered systemically or locally to the tumor or cancer site or affected organ.

In one aspect, the VLP is combined with and delivers or activates one or more toll-like receptor agonist, e.g. a TLR-2, 3, 4, 7, or 9 or a TLR-2, 3, 4, 7, or 9 agonist. In a further aspect, an immune checkpoint inhibitor is administered. Ultimately, this combined in situ vaccination approach reprograms the tumor microenvironment and incite systemic and durable antitumor immunity to protect patients from residual, metastatic and recurrent cancer. Without being bound by theory, the combination of ablation, e.g., cryoablation with VLP, e.g., CPMV activates the innate immune system through TLR 2, 3, 4, 7 and/or 9, leading to systemic immunity.

Thus, provided herein is a method of one or more of: treating or inhibiting the growth of a tumor or cancer in a subject in need thereof comprising administration of an effective amount of a naturally occurring plant virus or an engineered VLP and subsequent ablation, e.g., cryoablation, irreversible electroporation, radiofrequency ablation, or microwave ablation of the tumor or cancer, optionally wherein the cancer is localized or metastatic. Compositions for use in these methods are further provided herein.

### BRIEF DESCRIPTION OF THE DRAWINGS

FIGS. 1A-1B: The figure shows fold change in tumor growth at 2 weeks for (FIG. 1A) primary and (FIG. 1B) secondary HCC tumors in Group A (Control), Group B (CPMV only), Group C (Cryo Only) and Group D (both).

FIGS. 2A-2B: Serum concentrations of (FIG. 2A) IL-10 and (FIG. 2B) CXCL1 in the four experimental groups.

FIGS. 3A-3C: Flow cytometry analysis showing (FIG. 3A) activated CD8+/CD137+ T cells in primary and secondary tumors, (FIG. 3B) primary tumor CD3-natural killer (NK) cells and (FIG. 3C) PD-1 expression on CD4+ and CD8+ T cells.

FIG. 4: Depiction of the treatment schedule for the experiments described in FIGS. 5 to FIG. 11. The primary tumor is treated with either PBS (Control), CPMV, Cryo+PBS, or Cryo+CPMV. The secondary tumor is observed.

FIGS. 5A-5C depicts change in tumor volume with various treatments: (FIG. 5A) Fold change in primary tumor volume over 2 weeks compared to baseline volume. (FIG. 5B) Fold change in tumor size at 2 weeks post treatment initiation, CPMV, Cryo and Cryo+CPMV groups demonstrated significantly slower growth compared with the Control group (black). Cryo+CPMV resulted in the greatest reduction of tumor growth vs. Control (1.6±0.9 vs. 6.3±0.5, p<0.0001). (FIG. 5C) Variance of tumor volume was significantly lower in the Cryo+CPMV group compared to both CPMV and Cryo groups. Time course data were analyzed by repeated measures 2-way ANOVA, and fold-change in tumor size at 2-weeks was analyzed by 1-way ANOVA with Tukey ad hoc post tests. Asterisks indicate statistical significance as shown; *p<0.05, **p<0.01, ****p<0.0001.

FIGS. 6A-6B: (FIG. 6A) Fold change in untreated tumor volumes over time compared to baseline. (FIG. 6B) At 2 weeks post treatment initiation, Cryo+CPMV-treated tumors showed significantly less growth compared with Control. Time course data were analyzed by repeated measures 2-way ANOVA, and tumor size at 2-weeks was analyzed by 1-way ANOVA with Tukey ad hoc post tests. Asterisks indicate statistical significance as shown; * p<0.05.

FIGS. 7A-7B: Of the panel of serum cytokines and chemokines that were assayed, IL-10 and CXCL1 demonstrated significant differences. (FIG. 7A) IL-10 concentrations were significantly increased at day 3 in Cryo+CPMV-treated tumors compared with Cryo at day 3 (20.0±2.7 pg/mL vs. 9.6±1.0 pg/mL; p=0.03). This difference did not persist at day 6. (FIG. 7B) CXCL1 was significantly decreased in Cryo+CPMV-treated tumors compared with CPMV at day 6 (98.2±17.6 pg/mL vs. 189.0±16.6 pg/mL; p=0.01).

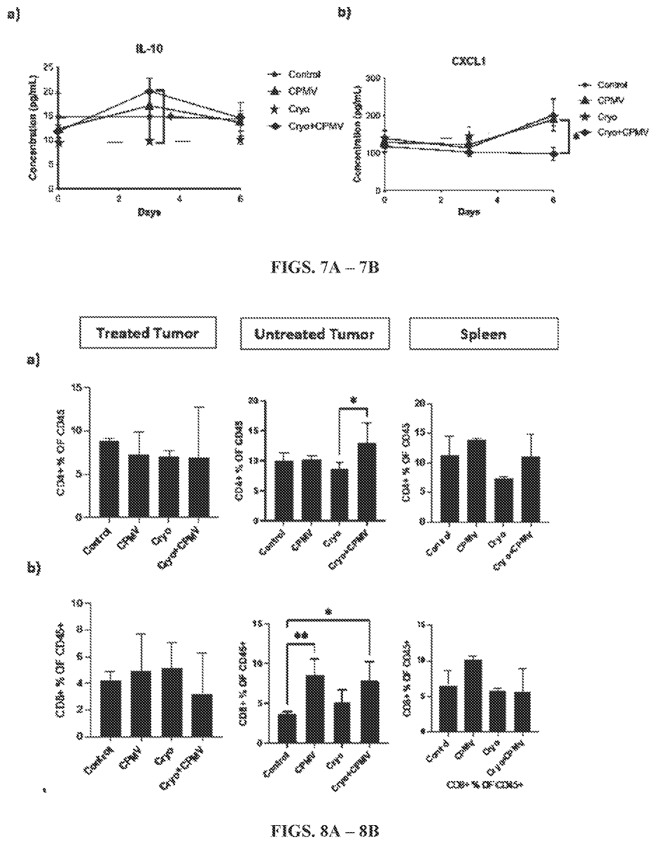

FIGS. 8A-8F: Flow cytometry of treated tumor, untreated tumor and spleen, showing percentage enrichment of (FIG. 8A) CD4+ T cells, (FIG. 8B) CD8+ T cells, (FIG. 8C) activated CD8+ T cells, (FIG. 8D) CD4+/PD-1+ T cells, (FIG. 8E) CD8+/PD-1+ T cells and (FIG. 8F) NK cells out of CD45 cells or CD8 cells. Immune cell enrichment was analyzed by 1-way ANOVA with Tukey ad hoc post tests. Asterisks indicate statistical significance as shown; * p<0.05, ** p<0.01.

FIGS. 9A-9F: Concentration of tumor-infiltrating CD4+ and CD8+lymphocytes in treated and untreated tumor IHC samples categorized by (FIG. 9A, FIG. 9B) overall, (FIG. 9C, FIG. 9D) peripheral and (FIG. 9E, FIG. 9F) central distributions. Cells were counted from 5-10 low-power fields from 3 sections from each mouse. Immune cell concentrations were analyzed by 1-way ANOVA with Tukey ad hoc post tests. Asterisks indicate statistical significance as shown; * p<0.05, **p<0.01, ***p<0.001, ****p<0.0001.

FIGS. 10A-10D: Representative IHC images of treated tumors at 5× magnification demonstrating CD4+ staining of (FIG. 10A) Control, (FIG. 10B) CPMV, (FIG. 10C) Cryo and (FIG. 10D) Cryo+CPMV. Control and CPMV tumors demonstrate CD4+ lymphocytes accumulation at the periphery of the tumors (brown stain) and a paucity of CD4+ cells in Cryo tumors. Cryo+CPMV tumors demonstrate increased concentration of CD4+ lymphocytes throughout the tumor, with diffuse fat necrosis (clear ovoid spaces).

FIG. 11: Percent change in weight from baseline. CPMV and Cryo+CPMV mice tended to weigh less than Control mice, but this difference did not reach statistical significance. Time course data were analyzed by repeated measures 2-way ANOVA.

### DETAILED DESCRIPTION

Unless defined otherwise, all technical and scientific terms used herein have the same meanings as commonly understood by one of ordinary skill in the art to which this disclosure belongs. All nucleotide sequences provided herein are presented in the 5′ to 3′ direction. All polypeptide and protein sequences are presented in the direction of the amine terminus to carboxy terminus. Although any methods and materials similar or equivalent to those described herein can be used in the practice or testing of the present disclosure, particular, non-limiting exemplary methods, devices, and materials are now described. All technical and patent publications cited herein are incorporated herein by reference in their entirety. Nothing herein is to be construed as an admission that the disclosure is not entitled to antedate such disclosure by virtue of prior invention.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds, (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration, and molecular weight, including ranges, are approximations which are varied (+) or (−) by increments of 1.0 or 0.1, as appropriate or alternatively by a variation of +/−15%, or alternatively 10% or alternatively 5% or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a polypeptide” includes a plurality of polypeptides, including mixtures thereof.

As used herein, “and/or” refers to and encompasses any and all possible combinations of one or more of the associated listed items, as well as the lack of combinations when interpreted in the alternative (“or”).

“Optional” or “optionally” means that the subsequently described circumstance may or may not occur, so that the description includes instances where the circumstance occurs and instances where it does not.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions disclosed herein. Embodiments defined by each of these transition terms are within the scope of this disclosure.

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) 15%, 10%, 5%, 3%, 2%, or 1%.

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

As used herein, the term “animal” refers to living multi-cellular vertebrate organisms, a category that includes, for example, mammals and birds. The term “mammal” includes both human and non-human mammals.

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, bonobos, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human. In some embodiments, a subject has or is diagnosed as having or is suspected of having a disease.

As used herein, the terms “treating,” “treatment” and the like are used herein to mean obtaining a desired pharmacologic and/or physiologic effect. In some embodiments, the effect can be prophylactic in terms of completely or partially preventing a disorder or sign or symptom thereof, and/or can be therapeutic in terms of a partial or complete cure for a disorder and/or adverse effect attributable to the disorder. Examples of “treatment” include but are not limited to: preventing a disorder from occurring in a subject that may be predisposed to a disorder, but has not yet been diagnosed as having it; inhibiting a disorder, i.e., arresting its development; and/or relieving or ameliorating the symptoms of disorder. In one aspect, treatment is the arrestment of the development of symptoms of the disease or disorder, e.g., a cancer such as breast cancer. In some embodiments, they refer to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and response (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor. In one aspect, treatment excludes prophylaxis.

In one embodiment, the term “disease” or “disorder” as used herein refers to a cancer or a tumor (which are used interchangeably herein), a status of being diagnosed with such disease, a status of being suspect of having such disease, or a status of at high risk of having such disease.

“Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features.

A “solid tumor” is an abnormal mass of tissue that usually does not contain cysts or liquid areas. Solid tumors can be benign or malignant. Different types of solid tumors are named for the type of cells that form them. Examples of solid tumors include sarcomas, carcinomas, and lymphomas. The solid tumor can be localized or metastatic.

The phrase “first-line” or “second-line” or “third-line” refers to the order of treatment received by a patient. First-line therapy regimens are treatments given first, whereas second- or third-line therapy are given after the first-line therapy or after the second-line therapy, respectively. The National Cancer Institute defines first-line therapy as “the first treatment for a disease or condition. In patients with cancer, primary treatment can be surgery, chemotherapy, radiation therapy, or a combination of these therapies. First-line therapy is also referred to those skilled in the art as “primary therapy and primary treatment”. Typically, a patient is given a subsequent chemotherapy regimen because the patient did not show a positive clinical or sub-clinical response to the first-line therapy or the first-line therapy has stopped.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and response (whether partial or total), whether detectable or undetectable. In one aspect, the term “treatment” excludes prevention.

Several classes of checkpoint inhibitors/regulators are known in the art, including lymphocyte activation gene-3 (LAG-3), T cell immunoglobulin and ITIM domain (TIGIT), T cell immunoglobulin and mucin-domain containing-3 (TIM-3), V-domain immunoglobulin suppressor of T cell activation (VISTA), B7 homolog 3 protein (B7-H3), inducible T cell costimulatory (ICOS) and B and T lymphocyte attenuator (BTLA), anti-cytotoxic T lymphocyte associated antigen-4 (CTLA-4) as well as inhibitors of CTLA-4, programmed death 1 (PD-1; also referred to herein as PD1), and programmed death ligand-1 (PD-L1). The latter 3 classes of checkpoint inhibitors, CTLA-4, PD-1 and PD-L1 inhibitors, have contributed several medically relevant drugs such as monoclonal antibody (mAb) checkpoint inhibitors. Example of anti-CTLA-4 checkpoint inhibitors include mAb such as ipilimumab (approved globally). Example of anti-PD-L1 checkpoint inhibitor include mAbs such as atezolizumab, avelumab and durvalumab (approved globally). Non-limiting examples of anti-PD1 checkpoint inhibitor therapies include mAbs such as pembrolizumab and nivolumab (approved globally); sintilimab, tislelizumab, toripalimab, and camrelizumab (approved in China); geptanolimab serplulimab zimberelimab cemiplimab, dostarlimab, prolgolimab, balstilimab, penpulimab, retifanlimab, cadonilimab, pucotenlimab, sasanlimab, and cetrelimab.

A toll-like receptor (TLR) refers to a class of proteins that play a key roll in the innate immune system. They are single-pass membrane-spanning receptors usually expressed on sentinel cells such as macrophages and dendritic cells, that recognize structurally conserved molecules derived from microbes. There are ten functional TLRs in human (TLR1-10) and twelve in mice (TLR1-9, 11-13). These are described in the literature, e.g., Medzhitov, Toll-like receptors and innate immunity, Nature Rev. Immun. 1:135-145 (2001); Nie et al., Toll-like receptors, associated biological roles and signaling networks in non-mammals, Front. Immunol., Jul. 2, 2018, https://doi.org/10.3389/fimmu.2018.01523, accessed Nov. 11, 2021, each incorporated herein by reference.

A “TLR agonist” is an agonist that targets TLR. Examples of such are known in the art. See, Bhardwaj, Cancer J. 2010 July-August; 16 (4): 382-391 and Kaczanowska, J. et al., Leukoc Biol. 2013 June 93 (6): 847-863, each incorporated herein by reference. See also, Table 1 reproduced from Kaczanowska et al. (2013).

| | Cellular | Adaptor | | Source of | |
|---|---|---|---|---|---|
| TLR | localization | molecule | Ligand/agonist | ligand | Clinical |
| TLR1- | Surface | MyD88 | Triacylated | Bacteria | BCG^{a} |
| TLR2 | | | lipoproteins, | | |
| | | | lipoteichoic | | |
| | | | acid, | | |
| | | | peptidoglycans | | |
| | | | Zymosan | Fungi | — |
| | | | Pam3CSK4 | Synthetic | — |
| TLR2- | Surface | MyD88 | Diacylated | Bacteria | BCGa |
| TLR6 | | | lipopeptides | | |
| | | | HSPs, HMGB1, | Endogenous | — |
| | | | uric acid, | | |
| | | | fibronectin, | | |
| | | | ECM proteins | | |
| | | | Pam₃CSK₄ | Synthetic | — |
| TLR3 | Endosome | TRIF | dsRNA | Virus | — |
| | | | Poly I:C | Synthetic | Poly A:U |
| TLR4 | Surface (or | MyD88 or | LPS, | Bacteria | BCGa |
| | endosome) | TRIF | lipoteichoic acid | | |
| | | | β-defensin 2, | Endogenous | |
| | | | fibronectin | | |
| | | | EDA, HMGB1, | | |
| | | | snapin, tenascin | | |
| | | | C | | |
| | | | | Synthetic | MPL^{a} |
| TLR5 | Surface | MyD88 | Flagellin | Bacteria | — |
| TLR7- | Endosome | MyD88 | ssRNA | Virus | — |
| TLR8 | | | CpG-A, Poly | Synthetic | Imiquimod |
| | | | G10, Poly G3 | | (Aldara)a |
| | | | | | 852A (Phase |
| | | | | | II) |
| TLR9 | Endosome | MyD88 | Unmethylated | Bacteria and | — |
| | | | CpG DNA | virus | |
| | | | | Bacteria | BCGa |
| | | | | Synthetic | EMD |
| | | | | | 120108 |
| | | | | | (Phase I) |
| | | | | | IMO-2055 |
| | | | | | (Phase II) |
| TLR10 | Surface | MyD88 | Unknown | Synthetic | — |
| | | | natural ligand | | |
| | | | Pam₃CSK₄, | | |
| | | | PamCysPamSK₄ | | |
| TLR11b | — | — | Toxoplasma | Protozoa | — |
| | | | gondii profiling | | |
| TLR12^{b} | — | — | Unknown | — | — |
| TLR13^{b} | Endosome | MyD88 | VSV | Virus | — |

TLRs are characterized by their cellular localization, adaptor proteins, and the PAMPs and DAMPs that they recognize. Agonists come from a variety of sources-natural and synthetic. Several TLR agonists are approved for clinical use, whereas others are being tested in clinical trials for their potential as anticancer therapies. With respect to the above table, ^{a}FDA-approved treatment for cancer; bexpression detected in mouse cells but not human cells.

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, glucose6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³²P, ³⁵S, ⁸⁹Zr or ¹²⁵I.

As used herein, the term “purification marker” refers to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly (NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double-and single-stranded molecules. Unless otherwise specified or required, any embodiment disclosed herein that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmid vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene. This is a cheap and easy way of mass-producing a gene or the protein it then codes for.

As used herein, the term “plant virus” includes viruses that infect plants or plant systems, e.g., leaves, root and/or stems. Plant viruses can be stably stored (and are stable without cold chain requirements). Plant viruses do not infect or replicate in mammalian cells, thus adding another layer of safety compared to oncolytic viral therapies. Non-limiting examples include tobacco mosaic virus (TMV), tobacco mold green mottle virus (TMGMV), physalis mottle virus like particle (PhMV), cowpea chlorotic mottle virus (CCMV), and cowpea mosaic virus (CPMV). Method of replicating and producing virus for therapeutic application are known in the art and described in WO 2022/221692, published Oct. 20, 2022, and incorporated herein by reference.

As used herein, the term “Virus-like particle” or “VLP” refers to a non-replicating, viral shell, derived from one or more plant viruses (e.g., one or more plant viruses described herein). VLPs are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system. VLPs can also be engineered, e.g., comprising, or consisting essentially of, or yet further consisting of, one or more viral proteins that comprise, or consists essentially of, or yet further consists of, a modification. Methods for producing VLPs are known in the art. The presence of VLPs following recombinant expression of viral proteins can be detected using conventional techniques known in the art, such as by electron microscopy, biophysical characterization, and the like. Further, VLPs can be isolated by known techniques, e.g., density gradient centrifugation and identified by characteristic density banding. See, for example, Baker et al. (1991) Biophys. J. 60:1445-1456; and Hagensee et al. (1994) J. Viral. 68:4503-4505; Vincente, J Invertebr Pathol., 2011; Schneider Ohrum and Ross, Curr. Top. Microbial. Immunol., 354:53073, 2012). As used herein, VLP intends naturally occurring (wild-type or native) VLP and engineered VLP, unless explicitly stated otherwise.

Virus and Virus-like Particles (VLPs) are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system. VLPs can also be engineered, e.g., comprising, or consisting essentially of, or yet further consisting of, one or more viral proteins that comprise, or consists essentially of, or yet further consists of, a modification. Methods for producing VLPs are known in the art. The presence of VLPs following recombinant expression of viral proteins can be detected using conventional techniques known in the art, such as by electron microscopy, biophysical characterization, and the like. Further, VLPs can be isolated by known techniques, e.g., density gradient centrifugation and identified by characteristic density banding. See, for example, Baker et al. (1991) Biophys. J. 60:1445-1456; and Hagensee et al. (1994) J. Viral. 68:4503-4505; Vincente, J Invertebr Pathol., 2011; Schneider Ohrum and Ross, Curr. Top. Microbial. Immunol., 354:53073, 2012).

In some embodiments, the virus or VLP is derived from Cowpea chlorotic mottle virus (CCMV). CCMV is a spherical plant virus that belongs to the Bromovirus genus. Several strains have been identified and include, but not limited to, Carl (Ali, et al., 2007. J. Virological Methods 141:84-86), Car2 (Ali, et al., 2007. J. Virological Methods 141:84-86, 2007), type T (Kuhn, 1964. Phytopathology 54:1441-1442), soybean(S) (Kuhn, 1968. Phytopathology 58:1441-1442), mild (M) (Kuhn, 1979. Phytopathology 69:621-624), Arkansas (A) (Fulton, et al., 1975. Phytopathology 65:741-742), bean yellow stipple (BYS) (Fulton, et al., 1975. Phytopathology 65:741-742), R (Sinclair, ed. 1982. Compendium of Soybean Diseases. 2^{nd} ed. The American Phytopathological Society, St. Paul. 104 pp.), and PSM (Paguio, et al., 1988. Plant Diseases 72 (9): 768-770).

In some instances, the virus or VLP from CCMV comprise, or consists essentially of, or yet further consists of, a plurality of capsid proteins. In some instances, the capsid protein is a wild-type CCMV capsid, optionally expressed by Car1, Car2, type T, soybean(S) , mild (M), Arkansas (A), bean yellow stipple (BYS), R, or PSM strain. In other instances, the capsid protein is a modified capsid protein, e.g., comprising, or consisting essentially of, or yet further consisting of, one or more substitutions, insertions, and/or deletions. In some cases, the CCMV capsid comprises, or consists essentially of, or yet further consists of, the sequence as set forth in the UniProtKB ID P03601:

(SEQ ID NO: )
MSTVGTGKLTRAQRRAAARKNKRNTRVVQPVIVEPIASGQGKAIKAWTG
YSVSKWTASCAAAEAKVTSAITISLPNELSSERNKQLKVGRVLLWLGLL
PSVSGTVKSCVTETQTTAAASFQVALAVADNSKDVVAAMYPEAFKGITL
EQLTADLTIYLYSSAALTEGDVIVHLEVEHVRPTFDDSFTPVY,
or
an equivalent thereof.

In some cases, the virus or VLP from CCMV is prepared by the method as described in Ali et al., “Rapid and efficient purification of Cowpea chlorotic mottle virus by sucrose cushion ultracentrifugation,” Journal of Virological Methods 141:84-86 (2007).

In some embodiments, the virus or VLP is derived from Cowpea mosaic virus(CPMV). CPMV is a non-enveloped plant virus that belongs to the Comovirus genus. CPMV strains include, but are not limited to, SB (Agrawal, H. O. (1964). Meded. Landb. Hoogesch. Wagen. 64:1) and Vu (Agrawal, H. O. (1964). Meded. Landb. Hoogesch. Wagen. 64:1). Cowpea mosaic virus (CPMV) is a VLP and a plant-infecting member of the order Picornavirales, with a relatively simple, non-enveloped capsid that has been extensively studied and a positive-sense, single-stranded RNA genome. For CPMV, the genome is bipartite, with RNA-1 (6 kb) and RNA-2 (3.5 kb) being separately encapsidated. CPMV has an icosahedral capsid structure, which is ˜30 nm in diameter and is formed from 60 copies each of a Large (L) and Small(S) coat protein. These two coat proteins are processed from a single RNA-2-encoded precursor polyprotein (VP60) by the action of the 24 K viral proteinase which is encoded by RNA-1. Thus capsid assembly, as well as viral infection, is dependent on the presence of both genomic segments in an infected plant cell. In some embodiments, the VLP particles have been treated, prepared and/or inactivated by methods known in the art. In some embodiments, the CPMV particle further comprises one or more TLR agonists, that may be the same or different. In some instances, the virus or VLP from CPMV comprise, or consists essentially of, or yet further consists of, a plurality of capsid proteins. In some instances, CPMV produces a large capsid protein and a small capsid protein precursor (which generates a mature small capsid protein). In some cases, CPMV capsid is formed from a plurality of large capsid proteins and mature small capsid proteins. In some cases, the large capsid protein is a wild-type large capsid protein, optionally expressed by SB or Vu strain. In other instances, the large capsid protein is a modified large capsid protein, e.g., comprising, or consisting essentially of, or yet further consisting of, one or more substitutions, insertions, and/or deletions. In some cases, the large capsid protein comprise, or consists essentially of, or yet further consists of, the sequence as set forth in the UniProtKB ID P03599 (residues 460-833):

(SEQ ID NO: )
MEQNLFALSLDDTSSVRGSLLDTKFAQTRVLLSKAMAGGDVLLDEYLYD
VVNGQDFRATVAFLRTHVITGKIKVTATTNISDNSGCCLMLAINSGVRG
KYSTDVYTICSQDSMTWNPGCKKNFSFTFNPNPCGDSWSAEMISRSRVR
MTVICVSGWTLSPTTDVIAKLDWSIVNEKCEPTIYHLADCQNWLPLNRW
MGKLTFPQGVTSEVRRMPLSIGGGAGATQAFLANMPNSWISMWRYFRGE
LHFEVTKMSSPYIKATVTFLIAFGNLSDAFGFYESFPHRIVQFAEVEEK
CTLVFSQQEFVTAWSTQVNPRTTLEADGCPYLYAIIHDSTTGTISGDFN
LGVKLVGIKDFCGIGSNPGIDGSRLLGAIAQ,
or
an equivalent thereof.

In some cases, the mature small capsid protein is a wild-type mature small capsid protein, optionally expressed by SB or Vu strain. In other instances, the mature small capsid protein is a modified mature small capsid protein, e.g., comprising, or consisting essentially of, or yet further consisting of, one or more substitutions, insertions, and/or deletions. In some cases, the mature small capsid protein comprise, or consists essentially of, or yet further consists of, the sequence as set forth in the UniProtKB ID P03599 (residues 834-1022):

(SEQ ID NO: )
GPVCAEASDVYSPCMIASTPPAPFSDVTAVTFDLINGKITPVGDDNWNT
HIYNPPIMNVLRTAAWKSGTIHVQLNVRGAGVKRADWDGQVFVYLRQSM
NPESYDARTFVISQPGSAMLNFSFDIIGPNSGFEFAESPWANQTTWYLE
CVATNPRQIQQFEVNMRFDPNFRVAGNILMPPFPLSTETPPL,
or
an equivalent thereof.

In some embodiments, the virus or VLP is derived from Physalis mottle virus(PhMV). PhMV is a single stranded RNA virus that belongs to the genus Tymovirus. In some instances, the virus or VLP from PhMV comprises, or consists essentially of, or yet further consists of, a plurality of coat proteins. In some instances, the coat protein is a wild-type PhMV coat protein. In other instances, the coat protein is a modified coat protein, e.g., comprising, or consisting essentially of, or yet further consisting of, one or more substitutions, insertions, and/or deletions. In some cases, the PhMV coat comprise, or consists essentially of, or yet further consists of, the sequence as set forth in the UniProtKB ID P36351:

(SEQ ID NO: )
MDSSEVVKVKQASIPAPGSILSQPNTEQSPAIVLPFQFEATTFGTAETA
AQVSLQTADPITKLTAPYRHAQIVECKAILTPTDLAVSNPLTVYLAWVP
ANSPATPTQILRVYGGQSFVLGGAISAAKTIEVPLNLDSVNRMLKDSVT
YTDTPKLLAYSRAPTNPSKIPTASIQISGRIRLSKPMLIAN,
or
an equivalent thereof.

In some embodiments, the virus or VLP is derived from Sesbania mosaic virus (SeMV). SeMV is a positive stranded RNA virus that belongs to the genus Sobemovirus. In some instances, the virus or VLP from SeMV comprise, or consists essentially of, or yet further consists of, a plurality of capsid proteins. In some instances, the capsid protein is a wild-type SeMV capsid protein. In other instances, the capsid protein is a modified capsid protein, e.g., comprising, or consisting essentially of, or yet further consisting of, one or more substitutions, insertions, and/or deletions. In some cases, the SeMV capsid comprises, or consists essentially of, or yet further consists of, the sequence as set forth in the UniProtKB ID Q9EB06:

(SEQ ID NO: )
MAKRLSKQQLAKAIANTLETPPQPKAGRRRNRRRQRSAVQQLQPTQAGI
SMAPSAQGAMVRIRNPAVSSSRGGITVLTHSELSAEIGVTDSIVVSSEL
VMPYTVGTWLRGVAANWSKYSWLSVRYTYIPSCPSSTAGSIHMGFQYDM
ADTVPVSVNQLSNLRGYVSGQVWSGSAGLCFINGTRCSDTSTAISTTLD
VSKLGKKWYPYKTSADYATAVGVDVNIATPLVPARLVIALLDGSSSTAV
AAGRIYCTYTIQMIEPTASALNN,
or
an equivalent thereof.

The terms “CPMV” “CPMV virus” or “CPMV particles” are used interchangeably, referring to a CPMV comprising, or alternatively consisting essentially of, or yet consisting of a capsid and an RNA genome (which is also referred to herein as a viral genome) encapsidated in the capsid. In some embodiments, the CPMV particles have been treated, prepared and/or inactivated by a method as disclosed herein. In some embodiments, the CPMV particle further comprises a heterologous RNA, which is heterologous to (i.e., not naturally presented in) a native CPMV free of any human intervention. In some embodiments, the CPMV particle further comprises one or more TLR agonists, that may be the same or different.

The virus can be obtained according to various methods known to those skilled in the art. In embodiments where plant virus particles are used, the virus particles can be obtained from the extract of a plant infected by the plant virus. For example, cowpea mosaic virus can be grown in black eyed pea plants, which can be infected within 10 days of sowing seeds. Plants can be infected by, for example, coating the leaves with a liquid containing the virus, and then rubbing the leaves, preferably in the presence of an abrasive powder which wounds the leaf surface to allow penetration of the leaf and infection of the plant. Within a week or two after infection, leaves are harvested and viral nanoparticles are extracted. In the case of cowpea mosaic virus, 100 mg of virus can be obtained from as few as 50 plants. Procedures for obtaining plant picornavirus particles using extraction of an infected plant are known to those skilled in the art. See Wellink J., Meth Mol Biol, 8, 205-209 (1998). Procedures are also available for obtaining virus-like particles. Saunders et al., Virology, 393 (2): 329-37(2009). The disclosures of both of these references are incorporated herein by reference.

As used herein, the term “cancer” refers to a plurality of cancer cells that may or may not be metastatic, such as prostate cancer, liver cancer, bladder cancer, skin cancer (e.g., cutaneous, melanoma, basal cell carcinoma, Kaposi's sarcoma, etc.), ovarian cancer, breast cancer, lung cancer, cervical cancer, pancreatic cancer, colon cancer, stomach cancer, esophagus cancer, mouth cancer, tongue cancer, gum cancer, muscle cancer, heart cancer, bronchial cancer, testis cancer, kidney cancer, endometrium cancer, and uterus cancer. Cancer may be a primary cancer, recurrent cancer, and/or metastatic cancer. The place where a cancer starts in the body is called the “primary cancer” or “primary site.” If cancer cells spread to another part of the body the new area of cancer is called a “secondary cancer” or a “metastasis.” “Recurrent cancer” means the presence of cancer after treatment and after a period of time during which the cancer cannot be detected. The same cancer may be detected at the primary site or somewhere else in the body, e.g., as a metastasis. In some embodiments, the cancer is an epithelial cancer. In further embodiments, the cancer is a mammary epithelial cancer. In some embodiments, the terms cancer and tumor are used interchangeably.

A cancer can be categorized in to various types, optionally based on its origin or location or both, such as those accessible at www.cancer.org/cancer/all-cancer-types.html or www.cancer.net/cancer-types or both. In some embodiments, the term “of the same cancer type” refers to cancer cells located in the same organ or tissue. In some embodiments, the term “of the same cancer type” refers to cancer cells of substantially the same morphology or phenotype or both.

In some embodiments, “a cancer cell” as used herein may refer to one or more cancer cell(s). Additionally or alternatively, “a cancer cell” as used herein may also refer to a progeny of the cancer cell, or a cell population comprising the cancer cell, or a cell population comprising the progeny, or a cell population comprising the cancer cell and the progeny thereof.

As used herein, the term “cancer cell” refers to a cell undergoing early, intermediate or advanced stages of multi-step neoplastic progression as previously described (Pitot et al., Fundamentals of Oncology, 15-28 (1978)). This includes cells in early, intermediate and advanced stages of neoplastic progression including “pre-neoplastic” cells (i.e., “hyperplastic” cells and dysplastic cells), and neoplastic cells in advanced stages of neoplastic progression of a dysplastic cell.

As used herein, a “metastatic” cancer cell refers to a cancer cell that is translocated from a primary cancer site (i.e., a location where the cancer cell initially formed from a normal, hyperplastic or dysplastic cell) to a site other than the primary site, where the translocated cancer cell lodges and proliferates.

As used herein, the terms “reduce” and “inhibit” are used together because it is recognized that, in some cases, a decrease can be reduced below the level of detection of a particular assay, for example by about 5%, about 10%, about 15%, about 20%, about 25%, about 30%, about 40%, about 50%, about 60%, about 70%, about 80%, about 85%, about 90%, about 95%, about 99%, or about 100% of the reference level. As such, it may not always be clear whether the expression level or activity is “reduced” below a level of detection of an assay, or is completely “inhibited.” Nevertheless, it will be clearly determinable, following a treatment according to the present methods.

The term “suitable for a therapy” or “suitably treated with a therapy” shall mean that the patient is likely to exhibit one or more desirable clinical outcomes as compared to patients having the same disease and receiving the same therapy but possessing a different characteristic that is under consideration for the purpose of the comparison. In one aspect, the characteristic under consideration is a genetic polymorphism or a somatic mutation. In another aspect, the characteristic under consideration is expression level of a gene or a polypeptide. In one aspect, a more desirable clinical outcome is relatively higher likelihood of or relatively better tumor response such as tumor load reduction. In another aspect, a more desirable clinical outcome is relatively longer overall survival. In yet another aspect, a more desirable clinical outcome is relatively longer progression free survival or time to tumor progression. In yet another aspect, a more desirable clinical outcome is relatively longer disease free survival. In further another aspect, a more desirable clinical outcome is relative reduction or delay in tumor recurrence. In another aspect, a more desirable clinical outcome is relatively decreased metastasis. In another aspect, a more desirable clinical outcome is relatively lower relative risk. In yet another aspect, a more desirable clinical outcome is relatively reduced toxicity or side effects. In some embodiments, more than one clinical outcomes are considered simultaneously. In one such aspect, a patient possessing a characteristic, such as a genotype of a genetic polymorphism, can exhibit more than one more desirable clinical outcomes as compared to patients having the same disease and receiving the same therapy but not possessing the characteristic. As defined herein, the patient is considered suitable for the therapy. In another such aspect, a patient possessing a characteristic can exhibit one or more desirable clinical outcome but simultaneously exhibit one or more less desirable clinical outcome. The clinical outcomes will then be considered collectively, and a decision as to whether the patient is suitable for the therapy will be made accordingly, taking into account the patient's specific situation and the relevance of the clinical outcomes. In some embodiments, progression free survival or overall survival is weighted more heavily than tumor response in a collective decision making.

A “tumor response” (TR) refers to a tumor's response to therapy. A “complete response” (CR) to a therapy refers to the clinical status of a patient with evaluable but non-measurable disease, whose tumor and all evidence of disease have disappeared following administration of the therapy. In this context, a “partial response” (PR) refers to a response that is anything less than a complete response. “Stable disease” (SD) indicates that the patient is stable following the therapy. “Progressive disease” (PD) indicates that the tumor has grown (i.e. become larger) or spread (i.e. metastasized to another tissue or organ) or the overall cancer has gotten worse following the therapy. For example, tumor growth of more than 20 percent since the start of therapy typically indicates progressive disease. “Non-response” (NR) to a therapy refers to status of a patient whose tumor or evidence of disease has remained constant or has progressed.

“Overall Survival” (OS) refers to the length of time of a cancer patient remaining alive following a cancer therapy.

“Progression free survival” (PFS) or “Time to Tumor Progression” (TTP) refers to the length of time following a therapy, during which the tumor in a cancer patient does not grow. Progression-free survival includes the amount of time a patient has experienced a complete response, partial response or stable disease.

“Disease free survival” refers to the length of time following a therapy, during which a cancer patient survives with no signs of the cancer or tumor.

“Time to Tumor Recurrence (TTR)” refers to the length of time, following a cancer therapy such as surgical resection or chemotherapy, until the tumor has reappeared (come back). The tumor may come back to the same place as the original (primary) tumor or to another place in the body.

As used herein, an anticancer agent refers to any drug or compound used for anticancer treatment. These include any drug that renders or maintains a clinical symptom or diagnostic marker of tumors and cancer, alone or in combination with other compounds, that reduces or maintains a state of remission, or reduction or prevention or remission. In some embodiments, the agent is an RNA and/or a DNA. In some embodiments, the agent is a protein or a polypeptide. In some embodiments, the agent is a chemical compound. Examples of anticancer agents include angiogenesis inhibitors such as angiostatin K1-3, DL-adifluoromethyl-ornithine, endostatin, fumagillin, genistein, minocycline, staurosporine, and (+)-thalidomide; DNA intercalating or cross-linking agents such as bleomycin, carboplatin, carmustine, chlorambucil, cyclophosphamide, cisplatin, melphalan, mitoxantrone, and oxaliplatin; DNA synthesis inhibitors such as methotrexate, 3-Amino-1,2,4-benzotriazine 1,4-dioxide, aminopterin, cytosine b-D-arabinofuranoside, 5-Fluoro-5′-deoxyuridine, 5-Fluorouracil, gaciclovir, hydroxyurea, and mitomycin C; DNA-RNA transcription regulators such as actinomycin D, daunorubicin, doxorubicin, homoharringtonine, and idarubicin; enzyme inhibitors such as S(+)-camptothecin, curcumin, (−)-deguelin, 5,6-dichlorobenzimidazole I-b-D-ribofuranoside, etoposine, formestane, fostriecin, hispidin, cyclocreatine, mevinolin, trichostatin A, tyrophostin AG 34, and tyrophostin AG 879, Gene Regulating agents such as 5-aza-2′-deoxycitidine, 5-azacytidine, cholecalciferol, 4-hydroxytamoxifen, melatonin, mifepristone, raloxifene, all trans-retinal, all trans retinoic acid, 9-cis-retinoic acid, retinol, tamoxifen, and troglitazone; Microtubule Inhibitors such as colchicine, dolostatin 15, nocodazole, paclitaxel, podophyllotoxin, rhizoxin, vinblastine, vincristine, vindesine, and vinorelbine; humanised or mouse/human chimeric monoclonal antibodies against defined cancer associated structures (such as Trastuzumab, Rituximab, Cetuximab, Bevacizumab, Alemtuzumab); and various other antitumor agents such as 17-(allylamino)-17-demethoxygeldanamycin, 4-Amino-1,8-naphthalimide, apigenin, brefeldin A, cimetidine, dichloromethylene-diphosphonic acid, leuprolide, luteinizing-hormone-releasing hormone, pifithrin-a, rapamycin, thapsigargin, and bikunin, and derivatives (as defined for imaging agents) thereof.

As used herein, “ablation” intends the removal or destruction of a part of a body or tissue, or its function. Ablation can be performed by surgery, hormones, drugs, radiofrequency, heat or other methods, e.g., cryoablation, irreversible electroporation, radiofrequency ablation, or microwave ablation. Cryoablation for cancer is a treatment to kill cancer cells with extreme cold. During cryoablation, a thin, wandlike needle (cryoprobe) is inserted through your skin and directly into the cancerous tumor. A gas is pumped into the cryoprobe in order to freeze the tissue. Then the tissue is allowed to thaw. A “composition” is intended to mean a combination of active agent and another compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers.

Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

As used herein, the term “contacting” means direct or indirect binding or interaction between two or more molecules. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include systemic or localized administration, e.g., oral administration, intratumorally, or localized at the site of the cancer or tumor, nasal administration, injection, and topical application. Additional non-limiting routes of administration include transdermal, intranasal, vaginal, rectal, subcutaneous intravenous, intravenous, intraarterial, intramuscular, intraosseous, intraperitoneal, intraocular, subconjunctival, sub-Tenon's, intravitreal, retrobulbar, intracameral, intratumoral, epidural and intrathecal.

An agent of the present disclosure can be administered for therapy by any suitable route of administration. It will also be appreciated that the optimal route will vary with the condition and age of the recipient, and the disease being treated.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. In general, one will desire to administer an amount of the compound that is effective to achieve a serum level commensurate with the concentrations found to be effective in vivo. These considerations, as well as effective formulations and administration procedures are well known in the art and are described in standard textbooks.

“Therapeutically effective amount” of a drug or an agent refers to an amount of the drug or the agent that is an amount sufficient to obtain a pharmacological response such as passive immunity; or alternatively, is an amount of the drug or agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose and may occur only after administration of a series of doses. Thus, a therapeutically effective amount may be administered in one or more administrations.

### Modes to Carry Out the Disclosure

Provided herein are compositions and methods that provide a novel, multimodal in situ vaccination approach to treat a cancer, e.g., hepatocellular carcinoma (HCC), by combining ablation such as percutaneous cryoablation with administration of VLP nanoparticles, e.g., cowpea mosaic virus (CPMV), a plant virus-based immunoadjuvant. The disclosed therapy is shown to be effective a subcutaneous mouse model of HCC. Cryoablation serves as a source of abundant intact tumor-associated antigens (TAA). As an example of the compositions and methods, CPMV is a multi-Toll like receptor (TLR) agonist that has been shown to reprogram the tumor microenvironment (TME) and recruit immune cells to process TAA and prime a durable, targeted anti-tumor immune response. In one aspect, the VLP further comprises one or more TLR agonists, that may be the same or different from each other.

CPMV signals through MyD88 and is a triple-pronged TLR agonist. Intratumoral CPMV modulates the TME to relieve immunosuppression. CPMV activates the innate immune cells (switch of M2 to M1 macrophages, Natural Killer (NK) cells, activation of immature dendritic cells (DCs) and N1 neutrophils) to initiate tumor cell killing and processing of tumor-associated antigens (TAAs). CPMV activates the innate immune system to kill tumor cells and process tumor antigens. These initial events lead to adaptive anti-tumor immunity against antigens expressed by the tumor. CPMV immunotherapy acts on the TME and leads to tumor antigen-specific CD4+ and CD8+ effector T cells and memory T cells. Therefore, CPMV-stimulated immune-mediated antitumor response is not only limited to injected tumors; efficacy against the primary tumor and distant metastatic sites has been reported; treatment protects from recurrence of the disease.

Applicant has determined that there is synergy between VLP and ablation such as cryoablation for the treatment of localized and metastatic cancer, e.g., HCC. Cryoblation kills tumor cells to release tumor antigens. VLP such as CPMV recruits innate immune cells to process the tumor antigens. Therefore, the therapeutic approach provides synergies.

### Compositions

Applicant provides herein a composition comprising, or consisting essentially of, or yet further consisting of a naturally occurring plant virus or engineered virus like particle (“VLP”) wherein the naturally occurring plant virus or VLP further comprises a toll-like receptor (“TLR”) agonist, or TLR agonist, optionally a TLR 2, 3, 4, 7 and/or 9 agonist, and optionally a carrier, and further optionally a detectable or purification label. The TLR agonist can be packaged within the naturally occurring plant virus or VLP. In another aspect, the naturally occurring plant virus or VLP is one that binds and activates the TLR in the absence of the TRL agonist.

Non-limiting examples of plant virus or the VLP are described herein. In one aspect, the plant virus or the VLP comprises cowpea mosaic virus (CPMV). In another aspect, the CPMV is naturally occurring CPMV.

The composition can further comprise a carrier, such as a pharmaceutically acceptable carriers, and or additional therapeutic agents for the treatment or detection of cancer or a tumor.

In one aspect, the composition comprises a plurality of the plant virus or VLP, that may be the same or different from each other in the composition. In another aspect, the plurality of plant virus or VLP are the same or different from each other in the composition. In a yet further aspect, the plurality of the TLR agonists of the plurality are the same or different from each other in the composition. In a yet further aspect, the plurality of the plant virus and/or the VLPs in the composition comprise different TLR agonists in the composition.

The compositions as described herein are useful in the methods as disclosed herein, and are useful in medical research, veterinary medicine and for the treatment of human patients in need of such therapy. Compositions, including pharmaceutical compositions comprising, consisting essentially of, or consisting of the engineered VLP alone or in combination with the TLR agonist and optionally the checkpoint inhibitor can be manufactured by means of conventional mixing, dissolving, granulating, dragee-making levigating, emulsifying, encapsulating, entrapping, or lyophilization processes. The compositions can be formulated in conventional manner using one or more physiologically acceptable carriers, diluents, excipients, or auxiliaries which facilitate processing of the combinations of compounds provided herein into preparations which can be used pharmaceutically.

In some embodiments, the pharmaceutical composition and formulations described herein are administered to a subject by multiple administration routes, including but not limited to, parenteral, oral, buccal, rectal, sublingual, or transdermal administration routes. In some cases, parenteral administration comprise, or consists essentially of, or yet further consists of, intravenous, intra-arterial, intradermal, subcutaneous, intramuscular, intracerebral, intranasal, inhaled, intra-articular, intravitreal, intraosseous, intraperitoneal, or intrathecal administration. In some instances, the pharmaceutical composition is formulated for local administration. In other instances, the pharmaceutical composition is formulated for systemic administration.

In some embodiments, the pharmaceutical formulations include, but are not limited to, lyophilized formulations, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995), Hoover, John E., Remington's Pharmaceutical Sciences, Mack Publishing Co., Easton, Pennsylvania 1975, Liberman, H. A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkins1999).

In some instances, the pharmaceutical formulations further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the pharmaceutical formulations include, but are not limited to, sugars like trehalose, sucrose, mannitol, maltose, glucose, or salts like potassium phosphate, sodium citrate, ammonium sulfate and/or other agents such as heparin to increase the solubility and in vivo stability of polypeptides.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as AVICEL®, dibasic calcium phosphate, dicalcium phosphate dihydrate, tricalcium phosphate, calcium phosphate, anhydrous lactose, spray-dried lactose, pregelatinized starch, compressible sugar, such as Di-PAC® (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some cases, the pharmaceutical formulations include disintegration agents or disintegrants to facilitate the breakup or disintegration of a substance. The term “disintegrate” includes both the dissolution and dispersion of the dosage form when contacted with gastrointestinal fluid. Examples of disintegration agents include a starch, e.g., a natural starch such as corn starch or potato starch, a pregelatinized starch such as National 1551 or AMIJEL®, or sodium starch glycolate such as PROMOGEL® or EXPLOTAB®, a cellulose such as a wood product, methylcrystalline cellulose, e.g., AVICEL®, AVICEL® PH101, AVICEL®PH102, AVICEL® PH105, ELCEMA® P100, EMCOCEL®, VIVACEL®, MING TIA®, and SOLKA-FLOC®, methylcellulose, croscarmellose, or a cross-linked cellulose, such as cross-linked sodium carboxymethylcellulose (AC-DI-SOLR), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as VEEGUM® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials.

Exemplary lubricants include, e.g., stearic acid, calcium hydroxide, talc, sodium stearyl fumerate, a hydrocarbon such as mineral oil, or hydrogenated vegetable oil such as hydrogenated soybean oil (STEROTEX®), higher fatty acids and their alkali-metal and alkaline earth metal salts, such as aluminum, calcium, magnesium, zinc, stearic acid, sodium stearates, glycerol, talc, waxes, STEAROWET®, boric acid, sodium benzoate, sodium acetate, sodium chloride, leucine, a polyethylene glycol (e.g., PEG-4000) or a methoxypolyethylene glycol such as CARBOWAX™, sodium oleate, sodium benzoate, glyceryl behenate, polyethylene glycol, magnesium or sodium lauryl sulfate, colloidal silica such as SYLOID™, CAB-O-SIL®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethyl citrate, ethyl oleate, ethyl caprylate, sodium lauryl sulfate, sodium docusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide, and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, poly sorb ate-20 or TWEEN® 20, or trometamol.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000 Da, or about 3350 to about 4000 Da, or about 7000 to about 5400 Da, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone, and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., PLURONIC® (BASF), and the like. Additional surfactants include polyoxyethylene fatty acid glycerides and vegetable oils, e.g., polyoxyethylene (60) hydrogenated castor oil, and polyoxyethylene alkyl ethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium docusate, triacetin, Tween-80, vitamin E TPGS, ammonium salts, and the like.

The pharmaceutical compositions for the administration of the combinations of compounds can be conveniently presented in dosage unit form and can be prepared by any of the methods well known in the art of pharmacy. The pharmaceutical compositions can be, for example, prepared by uniformly and intimately bringing the compounds provided herein into association with a liquid carrier, a finely divided solid carrier or both, and then, if necessary, shaping the product into the desired formulation. In the pharmaceutical composition, each compound of the combination provided herein is included in an amount sufficient to produce the desired therapeutic effect. For example, pharmaceutical compositions of the present technology may take a form suitable for virtually any mode of administration, including, for example, topical, ocular, oral, buccal, local, systemic, nasal, injection, infusion, transdermal, rectal, and vaginal, or a form suitable for administration by inhalation or insufflation. ®For topical administration, the combination of compounds can be formulated as solutions, gels, ointments, creams, suspensions, etc., as is well-known in the art.

Systemic formulations include those designed for administration by injection (e.g., subcutaneous, intravenous, intra-arterial, intramuscular, intrathecal, or intraperitoneal injection) as well as those designed for transdermal, transmucosal, oral, or pulmonary administration.

Useful injectable preparations include sterile suspensions, solutions, or emulsions of the compounds provided herein in aqueous or oily vehicles. The compositions may also contain formulating agents, such as suspending, stabilizing, and/or dispersing agents. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen-free water, buffer, and dextrose solution, before use. To this end, the combination of compounds provided herein can be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets, or capsules prepared by conventional means with pharmaceutically acceptable excipients such as binding agents (e.g., pregelatinized maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets can be coated by methods well known in the art with, for example, sugars, films, or enteric coatings.

Compositions intended for oral use can be prepared according to any method known to the art for the manufacture of pharmaceutical compositions, and such compositions may contain one or more agents selected from the group consisting of sweetening agents, flavoring agents, coloring agents, and preserving agents in order to provide pharmaceutically elegant and palatable preparations. Tablets contain the combination of compounds provided herein in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g. starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques well known to the skilled artisan. The pharmaceutical compositions of the present technology may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non aqueous vehicles (e.g., almond oil, oily esters, ethyl alcohol, cremophore™, or fractionated vegetable oils); and preservatives (e.g., methyl or propyl p hydroxybenzoates or sorbic acid). The preparations may also contain buffer salts, preservatives, flavoring, coloring, and sweetening agents as appropriate.

In some embodiments, one or more compositions disclosed herein are contained in a kit. Accordingly, in some embodiments, provided herein is a kit comprising, consisting essentially of, or consisting of one or more compositions disclosed herein and instructions for their use.

### Methods

In one aspect, provide herein is a method of one or more of: treating or inhibiting the growth of a tumor or cancer in a subject in need thereof, the method comprising, or alternatively consisting essentially or, or yet further consisting of, administration of an effective amount of a naturally occurring plant, a VLP or an engineered VLP or a composition comprising the naturally occurring plant virus, VLP or the engineered VLP, e.g., a cowpea mosaic virus (CPMV), and ablation, e.g., by a method comprising one or more of cryoablation, irreversible electroporation, radiofrequency ablation, or microwave ablation, e.g., subsequent cryoablation, percutaneous cryoablation, of the tumor or cancer in the subject. The cancer or tumor can be localized or metastatic. The administration is systemic or local to the tumor or cancer or affected organ in the subject.

In one aspect, the naturally occurring plant virus, the VLP or the engineered VLP further comprises a TLR, e.g. a TLR-2, 3, 4, 7 or 9 agonist. The TLR agonist can be packaged within or attached to the naturally occurring plant, the VLP, or the engineered VLP.

In another aspect, the naturally occurring plant virus, the VLP or the engineered VLP binds and activates the TLR in the absence of a TLR agonist. A non-limiting examples of such includes CPMV.

Also provided are the methods as disclosed herein wherein a plurality of the naturally occurring plant virus, the VLP or the engineered VLP are administered to the subject. In one aspect, the plurality that are administered comprise the same of different naturally occurring plant virus, the VLP or the engineered VLP that can contain the same or different TLR agonist. In one aspect, the VLP comprises CPMV and contains the same or different TLR agonist.

Methods to make VLP comprising molecules such as TLR agonists are known in the art, e.g. WO 2021/108202, incorporated herein by reference.

As utilized herein, an engineered VLP is a non-native or not naturally occurring VLP that comprise, or consists essentially of, or yet further consists of, one or more viral particles, e.g., a capsid, derived from a plant virus. In some instances, the plant virus is from the genus Bromovirus, Comovirus, Tymovirus, or Sobemovirus. In some cases, the VLP is derived from Cowpea chlorotic mottle virus (CCMV), Cowpea mosaic virus (CPMV), Physalis mottle virus (PhMV), or Sesbania mosaic virus (SeMV).

In some instances, the engineered VLP comprise, or consists essentially of, or yet further consists of, a capsid protein derived from a plant virus. In some instances, the capsid protein is a wild-type protein derived from the plant virus. In other instances, the capsid protein is a variant of the wild-type protein derived from the plant virus. In additional instances, the capsid protein is a modified protein, either full-length or truncated version.

In some embodiments, the engineered VLP is derived from Cowpea chlorotic mottle virus (CCMV). CCMV is a spherical plant virus that belongs to the Bromovirus genus. Several strains have been identified and include, but not limited to, Carl (Ali, et al., 2007. J. Virological Methods 141:84-86), Car2 (Ali, et al., 2007. J. Virological Methods 141:84-86, 2007), type T (Kuhn, 1964. Phytopathology 54:1441-1442), soybean(S) (Kuhn, 1968. Phytopathology 58:1441-1442), mild (M) (Kuhn, 1979. Phytopathology 69:621-624), Arkansas (A) (Fulton, et al., 1975. Phytopathology 65:741-742), bean yellow stipple (BYS) (Fulton, et al., 1975. Phytopathology 65:741-742), R (Sinclair, ed. 1982. Compendium of Soybean Diseases. 2^{nd} ed. The American Phytopathological Society, St. Paul. 104 pp.), and PSM (Paguio, et al., 1988. Plant Diseases 72 (9): 768-770).

In some instances, the engineered VLP from CCMV comprise, or consists essentially of, or yet further consists of, a plurality of capsid proteins. In some instances, the capsid protein is a wild-type CCMV capsid, optionally expressed by Car1, Car2, type T, soybean(S) , mild (M), Arkansas (A), bean yellow stipple (BYS), R, or PSM strain. In other instances, the capsid protein is a modified capsid protein, e.g., comprising, or consisting essentially of, or yet further consisting of, one or more substitutions, insertions, and/or deletions. In some cases, the CCMV capsid comprises, or consists essentially of, or yet further consists of, the sequence as set forth in the UniProtKB ID P03601:

(SEQ ID NO: )
MSTVGTGKLTRAQRRAAARKNKRNTRVVQPVIVEPIASGQGKAIKAWTG
YSVSKWTASCAAAEAKVTSAITISLPNELSSERNKQLKVGRVLLWLGLL
PSVSGTVKSCVTETQTTAAASFQVALAVADNSKDVVAAMYPEAFKGITL
EQLTADLTIYLYSSAALTEGDVIVHLEVEHVRPTFDDSFTPVY,
or
an equivalent thereof.

In some cases, the engineered VLP from CCMV is prepared by the method as described in Ali et al., “Rapid and efficient purification of Cowpea chlorotic mottle virus by sucrose cushion ultracentrifugation,” Journal of Virological Methods 141:84-86 (2007).

In some embodiments, the engineered VLP is derived from Cowpea mosaic virus(CPMV). CPMV is a non-enveloped plant virus that belongs to the Comovirus genus. CPMV strains include, but are not limited to, SB (Agrawal, H. O. (1964). Meded. Landb. Hoogesch. Wagen. 64:1) and Vu (Agrawal, H. O. (1964). Meded. Landb. Hoogesch. Wagen. 64:1).

In some instances, the engineered VLP from CPMV comprise, or consists essentially of, or yet further consists of, a plurality of capsid proteins. In some instances, CPMV produces a large capsid protein and a small capsid protein precursor (which generates a mature small capsid protein). In some cases, CPMV capsid is formed from a plurality of large capsid proteins and mature small capsid proteins. In some cases, the large capsid protein is a wild-type large capsid protein, optionally expressed by SB or Vu strain. In other instances, the large capsid protein is a modified large capsid protein, e.g., comprising, or consisting essentially of, or yet further consisting of, one or more substitutions, insertions, and/or deletions. In some cases, the large capsid protein comprise, or consists essentially of, or yet further consists of, the sequence as set forth in the UniProtKB ID P03599 (residues 460-833):

(SEQ ID NO: )
MEQNLFALSLDDTSSVRGSLLDTKFAQTRVLLSKAMAGGDVLLDEYLYD
VVNGQDFRATVAFLRTHVITGKIKVTATTNISDNSGCCLMLAINSGVRG
KYSTDVYTICSQDSMTWNPGCKKNFSFTFNPNPCGDSWSAEMISRSRVR
MTVICVSGWTLSPTTDVIAKLDWSIVNEKCEPTIYHLADCQNWLPLNRW
MGKLTFPQGVTSEVRRMPLSIGGGAGATQAFLANMPNSWISMWRYFRGE
LHFEVTKMSSPYIKATVTFLIAFGNLSDAFGFYESFPHRIVQFAEVEEK
CTLVFSQQEFVTAWSTQVNPRTTLEADGCPYLYAIIHDSTTGTISGDFN
LGVKLVGIKDFCGIGSNPGIDGSRLLGAIAQ,
or
an equivalent thereof.

In some cases, the mature small capsid protein is a wild-type mature small capsid protein, optionally expressed by SB or Vu strain. In other instances, the mature small capsid protein is a modified mature small capsid protein, e.g., comprising, or consisting essentially of, or yet further consisting of, one or more substitutions, insertions, and/or deletions. In some cases, the mature small capsid protein comprises, or consists essentially of, or yet further consists of, the sequence as set forth in the UniProtKB ID P03599 (residues 834-1022):

(SEQ ID NO: )
GPVCAEASDVYSPCMIASTPPAPFSDVTAVTFDLINGKITPVGDDNWNT
HIYNPPIMNVLRTAAWKSGTIHVQLNVRGAGVKRADWDGQVFVYLRQSM
NPESYDARTFVISQPGSAMLNFSFDIIGPNSGFEFAESPWANQTTWYLE
CVATNPRQIQQFEVNMRFDPNFRVAGNILMPPFPLSTETPPL,
or
an equivalent thereof.

In some embodiments, the engineered VLP is derived from Physalis mottle virus (PhMV). PhMV is a single stranded RNA virus that belongs to the genus Tymovirus. In some instances, the engineered VLP from PhMV comprises, or consists essentially of, or yet further consists of, a plurality of coat proteins. In some instances, the coat protein is a wild-type PhMV coat protein. In other instances, the coat protein is a modified coat protein, e.g., comprising, or consisting essentially of, or yet further consisting of, one or more substitutions, insertions, and/or deletions. In some cases, the PhMV coat comprises, or consists essentially of, or yet further consists of, the sequence as set forth in the UniProtKB ID P36351:

(SEQ ID NO: 19)
MDSSEVVKVKQASIPAPGSILSQPNTEQSPAIVLPFQFEATTFGTAETA
AQVSLQTADPITKLTAPYRHAQIVECKAILTPTDLAVSNPLTVYLAWVP
ANSPATPTQILRVYGGQSFVLGGAISAAKTIEVPLNLDSVNRMLKDSVT
YTDTPKLLAYSRAPTNPSKIPTASIQISGRIRLSKPMLIAN,
or
an equivalent thereof.

In some embodiments, the engineered VLP is derived from Sesbania mosaic virus (SeMV). SeMV is a positive stranded RNA virus that belongs to the genus Sobemovirus. In some instances, the engineered VLP from SeMV comprise, or consists essentially of, or yet further consists of, a plurality of capsid proteins. In some instances, the capsid protein is a wild-type SeMV capsid protein. In other instances, the capsid protein is a modified capsid protein, e.g., comprising, or consisting essentially of, or yet further consisting of, one or more substitutions, insertions, and/or deletions. In some cases, the SeMV capsid comprises, or consists essentially of, or yet further consists of, the sequence as set forth in the UniProtKB ID Q9EB06:

(SEQ ID NO: 20)
MAKRLSKQQLAKAIANTLETPPQPKAGRRRNRRRQRSAVQQLQPTQAGI
SMAPSAQGAMVRIRNPAVSSSRGGITVLTHSELSAEIGVTDSIVVSSEL
VMPYTVGTWLRGVAANWSKYSWLSVRYTYIPSCPSSTAGSIHMGFQYDM
ADTVPVSVNQLSNLRGYVSGQVWSGSAGLCFINGTRCSDTSTAISTTLD
VSKLGKKWYPYKTSADYATAVGVDVNIATPLVPARLVIALLDGSSSTAV
AAGRIYCTYTIQMIEPTASALNN,
or
an equivalent thereof.

As used herein, the term “an equivalent thereof” in reference to a polynucleotide or a protein (e.g., a capsid or coat protein) include a polynucleotide or a protein that comprise, or consists essentially of, or yet further consists of, at least 80%, 85%, 90%, 95%, 96%, 97%, 98%, or 99% sequence identify to the respective polynucleotide or protein of which it is compared to, while still retaining a functional activity. In the instances with reference to a capsid or coat protein, a functional activity refers to the formation of a VLP.

As used herein, the term “modification” include, for example, substitutions, additions, insertions and deletions to the amino acid sequences, which can be referred to as “variants.” Exemplary sequence substitutions, additions, and insertions include a full length or a portion of a sequence with one or more amino acids substituted (or mutated), added, or inserted, for example of a capsid derived from the plant virus. In some instances, a capsid described herein includes, e.g., a modified capsid comprising, or consisting essentially of, or yet further consisting of, at least 70%, 80%, 85%, 90%, 95%, 96%, 97%, 98%, 99%, or 100% sequence identity to its respective wild-type version.

The term “sequence identity” refers to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared. A polynucleotide or polynucleotide region (or a polypeptide or polypeptide region) having a certain percentage (for example, 60%, 65%, 70%, 75%, 80%, 85%, 90%, 95%, 98% or 99%) of “sequence identity” to a reference sequence means that, when aligned, that percentage of bases (or amino acids) at each position in the test sequence are identical to the base (or amino acid) at the same position in the reference sequence. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/blast/Blast.cgi.

Modified capsid polypeptides include, for example, non-conservative and conservative substitutions of the capsid amino acid sequences.

As used herein, the term “conservative substitution” denotes the replacement of an amino acid residue by another, chemically or biologically similar residue. Biologically similar means that the substitution does not destroy a biological activity or function, e.g., assembly of a viral capsid.

Structurally similar means that the amino acids have side chains with similar length, such as alanine, glycine and serine, or a similar size. Chemical similarity means that the residues have the same charge or are both hydrophilic or hydrophobic. Particular examples of conservative substitutions include the substitution of a hydrophobic residue such as isoleucine, valine, leucine or methionine for another, the substitution of a polar residue for another, such as the substitution of arginine for lysine, glutamic for aspartic acids, or glutamine for asparagine, and the like. The term “conservative substitution” also includes the use of a substituted amino acid in place of an unsubstituted parent amino acid. Such proteins that include amino acid substitutions can be encoded by a nucleic acid. Consequently, nucleic acid sequences encoding proteins that include amino acid substitutions are also provided.

Modified proteins also include one or more D-amino acids substituted for L-amino acids (and mixtures thereof), structural and functional analogues, for example, peptidomimetics having synthetic or non-natural amino acids or amino acid analogues and derivatized forms. Modifications include cyclic structures such as an end-to-end amide bond between the amino and carboxy-terminus of the molecule or intra-or inter-molecular disulfide bond.

Modified forms further include “chemical derivatives,” in which one or more amino acids has a side chain chemically altered or derivatized. Such derivatized polypeptides include, for example, amino acids in which free amino groups form amine hydrochlorides, p-toluene sulfonyl groups, carobenzoxy groups; the free carboxy groups form salts, methyl and ethyl esters; free hydroxl groups that form O-acyl or O-alkyl derivatives as well as naturally occurring amino acid derivatives, for example, 4-hydroxyproline, for proline, 5-hydroxylysine for lysine, homoserine for serine, ornithine for lysine etc. Also included are amino acid derivatives that can alter covalent bonding, for example, the disulfide linkage that forms between two cysteine residues that produces a cyclized polypeptide.

In some embodiments, the naturally occurring plant virus, the VLP or the engineered VLP further comprises a TLR agonist. In some cases, the TLR agonist comprises, or consists essentially of, or yet further consists of, a TLR1, TLR2, TLR3, TLR4, TLR5, TLR6, TLR7, TLR8, or TLR9 agonist. In some cases, the TLR agonist comprises, or consists essentially of, or yet further consists of, a synthetic ligand such as, for example, Pam3Cys, CFA, MALP2, Pam2Cys, FSL-1, Hib-OMPC, Poly I: C, poly A: U, AGP, MPL A, RC-529, MDF2p, CFA, or Flagellin. In another aspect, they are formulated as a ratio. In some instances, the ratio is a w/w ratio. In some instances, the engineered VLP and TLR agonist are formulated as a protein/TLR agonist (w/w) ratio of 1:1, 2:1, 3:1, 4:1, 5:1, 6:1, 7:1, 8:1, 9:1, 10:1, 1:2, 1:3, 1:4, 1:5, 1:6, 1:7, 1:8, 1:9, or 1:10. In some cases, the engineered VLP and TLR agonist are formulated as a protein/TLR agonist (w/w) ratio of 1:1. In some cases, the engineered VLP and TLR agonist are formulated as a protein/TLR agonist (w/w) ratio of 2:1. In some cases, the engineered VLP and TLR agonist are formulated as a protein/TLR agonist (w/w) ratio of 3:1. In some cases, the engineered VLP and TLR agonist are formulated as a protein/TLR agonist (w/w) ratio of 4:1. In some cases, the engineered VLP and TLR agonist are formulated as a protein/TLR agonist (w/w) ratio of 5:1. In some cases, the engineered VLP and TLR agonist are formulated as a protein/TLR agonist (w/w) ratio of 6:1. In some cases, the engineered VLP and TLR agonist are formulated as a protein/TLR agonist (w/w) ratio of 7:1. In some cases, the engineered VLP and TLR agonist are formulated as a protein/TLR agonist (w/w) ratio of 8:1. In some cases, the engineered VLP and TLR agonist are formulated as a protein/TLR agonist (w/w) ratio of 9:1. In some cases, the engineered VLP and TLR agonist are formulated as a protein/TLR agonist (w/w) ratio of 10:1.

In some instances, an engineered VLP described herein further comprise, or consists essentially of, or yet further consists of, a label or a tag, e.g., such as a detectable label. A detectable label can be attached to, e.g., to the surface of a VLP or to a TLR agonist encapsulated within the VLP.

Non-limiting exemplary detectable labels also include a radioactive material, such as a radioisotope, a metal or a metal oxide. Radioisotopes include radionuclides emitting alpha, beta or gamma radiation. In particular embodiments, a radioisotope can be one or more of: ³H, ¹⁰B, ¹⁸F, ¹¹C, ¹⁴C, ¹³N, ¹⁸O, ¹⁵O, ³²P, P^{33, 35}S, ³⁵Cl, ⁴⁵Ti, ⁴⁶Sc, ⁴⁷Sc, ⁵¹Cr, ⁵²Fe, ⁵⁹Fe, ^{0.57}Co, ⁶⁰Cu, ⁶¹Cu, ⁶²Cu, ⁶⁴Cu, ⁶⁷Cu, ⁶⁷Ga, ⁶⁸Ga, ⁷² As ⁷⁶Br, ⁷⁷Br, ⁸¹mKr, ⁸²Rb, ⁸⁵Sr, ⁸⁹Sr, ⁸⁶Y, ⁹⁰Y, ⁹⁵Nb, ⁹⁴mTc, ⁹⁹mTc, ⁹⁷Ru, ¹⁰³Ru, ¹⁰⁵Rh, ¹⁰⁹Cd, ¹¹¹In, ¹¹³Sn, ¹¹³mIn, ¹¹⁴In, I¹²⁵, I¹³¹, ¹⁴⁰La, ¹⁴¹Ce, ¹⁴⁹Pm, ¹⁵³Gd, ¹⁵⁷Gd, ¹⁵³Sm, ¹⁶¹Tb, ¹⁶⁶Dy, ¹⁶⁶Ho, ¹⁶⁹Er, ¹⁶⁹Y, ¹⁷⁵Yb, ¹⁷⁷Lu, ¹⁸⁶Re, ¹⁸⁸Re, ²⁰¹Tl, ²⁰³Pb, ²¹¹At, ²¹²Bi or ²²⁵Ac.

Additional non-limiting exemplary detectable labels include a metal or a metal oxide. In particular embodiments, a metal or metal oxide is one or more of: gold, silver, copper, boron, manganese, gadolinium, iron, chromium, barium, europium, erbium, praseodynium, indium, or technetium. In additional embodiments, a metal oxide includes one or more of: Gd(III), Mn(II), Mn(III), Cr(II), Cr(III), Cu(II), Fe(III), Pr(III), Nd(III) Sm(III), Tb(III), Yb(III) Dy(III), Ho(III), Eu(II), Eu(III), or Er(III).

Further non-limiting exemplary detectable labels include contrast agents (e.g., gadolinium; manganese; barium sulfate; an iodinated or noniodinated agent; an ionic agent or nonionic agent); magnetic and paramagnetic agents (e.g., iron-oxide chelate); nanoparticles; an enzyme (horseradish peroxidase, alkaline phosphatase, β-galactosidase, or acetylcholinesterase); a prosthetic group (e.g., streptavidin/biotin and avidin/biotin); a fluorescent material (e.g., umbelliferone, fluorescein, fluorescein isothiocyanate, rhodamine, dichlorotriazinylamine fluorescein, dansyl chloride or phycoerythrin); a luminescent material (e.g., luminol); or a bioluminescent material (e.g., luciferase, luciferin, aequorin).

Additional non-limiting examples of tags and/or detectable labels include enzymes (horseradish peroxidase, urease, catalase, alkaline phosphatase, beta-galactosidase, chloramphenicol transferase); enzyme substrates; ligands (e.g., biotin); receptors (avidin); GST-, T7-, His-, myc-, HA-and FLAG®-tags; electron-dense reagents; energy transfer molecules; paramagnetic labels; fluorophores (fluorescein, fluorscamine, rhodamine, phycoerthrin, phycocyanin, allophycocyanin); chromophores; chemi-luminescent (imidazole, luciferase, acridinium, oxalate); and bio-luminescent agents.

As set forth herein, a detectable label or tag can be linked or conjugated (e.g., covalently) to the VLP or TLR agonist. In various embodiments a detectable label, such as a radionuclide or metal or metal oxide can be bound or conjugated to the agent, either directly or indirectly. A linker or an intermediary functional group can be used to link the molecule to a detectable label or tag. Linkers include amino acid or peptidomimetic sequences inserted between the molecule and a label or tag so that the two entities maintain, at least in part, a distinct function or activity. Linkers may have one or more properties that include a flexible conformation, an inability to form an ordered secondary structure or a hydrophobic or charged character which could promote or interact with either domain. Amino acids typically found in flexible protein regions include Gly, Asn and Ser. The length of the linker sequence may vary without significantly affecting a function or activity.

Linkers further include chemical moieties, conjugating agents, and intermediary functional groups. Examples include moieties that react with free or semi-free amines, oxygen, sulfur, hydroxy or carboxy groups. Such functional groups therefore include mono and bifunctional crosslinkers, such as sulfo-succinimidyl derivatives (sulfo-SMCC, sulfo-SMPB), in particular, disuccinimidyl suberate (DSS), BS3 (Sulfo-DSS), disuccinimidyl glutarate (DSG) and disuccinimidyl tartrate (DST). Non-limiting examples include diethylenetriaminepentaacetic acid (DTPA) and ethylene diaminetetracetic acid. The tumor to be treated can be a solid tumor, e.g., a sarcoma, a carcinoma or a lymphoma. It can be localized or metastatic. In another aspect, the solid tumor cancer is selected from a cancer of the bladder, breast, cervix, colon, rectum, endometrium, kidney, lip, skin, oral cavity, liver, mesothelioma, lung, ovary, pancreas, and thyroid, that is local or metastatic. In another aspect, the method is used to treat a solid tumor, optionally a cancer of a cancer of an organ selected from the group of: the bladder, breast, cervix, colon, rectum, endometrium, kidney, esophagus, gastric tissue, adrenal gland, prostate, head and neck, mesothelium, skin, brain, oropharynx, stomach, liver, pancreas, ovaries, uterus, peritoneum, testes, lung, thyroid, or lymph nodes. In a further aspect, the tumor or cancer is a cancer of one or more of: the skin, central nervous system, head and neck, breast, thorax, digestive system, genitourinary system, pelvic organs, or lymphatic system.

In another aspect, the cancer is selected from hepatocellular carcinoma, non-small cell lung cancer, melanoma, and small cell lung cancer, that is local or metastatic.

The methods are useful to treat a subject in need of such treatment, having or suspected of having a cancer or tumor, e.g., an animal, mammal or a human patient.

The administered naturally occurring plant virus, VLPs or engineered VLPs, e.g., CPMV,s are typically administered in combination with a carrier such as a pharmaceutically acceptable carrier and the naturally occurring plant virus, VLPs or engineered VLP and/or CPMV can be the same or different from each other. In another aspect, provided is a composition comprising a VLPs, e.g., CPMV and a carrier such as a pharmaceutically acceptable carrier. In one aspect, the VLP further comprises a TLR and/or a TLR agonist, e.g. a TLR-2, 3, 4 7 or 9 or a TLR-2, 3, 4, 7 or 9, agonist. The TLR agonist can be the same or different from each other or the VLPs can be administered as a plurality of nanoparticles wherein the VLPs and TLR agonists are the same or different from each other. The TLR agonist can be contained within the plant virus, VLP or engineered VLP or attached to the particle of virus.

In another aspect, the method further comprises two or more subsequent ablations, that may be the same or different from each other. In a further aspect, the method further comprises administration of another anti-cancer therapy, such as for example, administration of an effective amount of a checkpoint inhibitor therapy to the subject. Examples of such checkpoint inhibitor therapy are known in the art, such as anti-PD-1 therapy.

The methods of this disclosure can be administered to the subject as a first-line, second-line, third-line, fourth-line, or fifth-line therapy.

Methods to determine if the therapies have been successful are known in the art and include one or more of: a reduction in tumor burden, increased time to or lack of tumor recurrence, complete or partial response, longer progression-free survival, longer disease-free survival, a better objective response rate, lowered toxicity or side effects, or increased overall survival, as compared to a subject who has not received the treatment.

### Dosage and Dosage Formulations

In some embodiments, the compositions may be administered to a subject suffering from a condition as disclosed herein, such as a human, either alone or as part of a pharmaceutically acceptable formulation, once a week, once a day, twice a day, three times a day, or four times a day, or even more frequently.

Administration of the VLP or engineered VLPs alone or in combination with the additional TLR agonist and/or checkpoint inhibitor therapy and compositions containing same can be effected by any method that enables delivery to the site of action. These methods include oral routes, intraduodenal routes, parenteral injection (including intravenous, subcutaneous, intramuscular, intravascular or infusion), topical, and rectal administration. Bolus doses can be used, or infusions over a period of 1, 2, 3, 4, 5, 10, 15, 20, 30, 60, 90, 120 or more minutes, or any intermediate time period can also be used, as can infusions lasting 3, 4, 5, 6, 7, 8, 9, 10, 12, 14 16, 20, 24 or more hours or lasting for 1-7 days or more. Infusions can be administered by drip, continuous infusion, infusion pump, metering pump, depot formulation, or any other suitable means.

Dosage regimens can be adjusted to provide the optimum desired response. For example, a single bolus can be administered, several divided doses can be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the subjects to be treated; each unit containing a predetermined quantity of active compound calculated to produce the desired therapeutic effect in association with the required pharmaceutical carrier. The specification for the dosage unit forms of the disclosure are dictated by and directly dependent on (a) the unique characteristics of the agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient can also be determined, as can the temporal requirements for administering each agent to provide a detectable therapeutic benefit to the patient. Accordingly, while certain dose and administration regimens are exemplified herein, these examples in no way limit the dose and administration regimen that can be provided to a patient in practicing the present disclosure.

It is to be noted that dosage values can vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration are well-known in the relevant art and would be understood to be encompassed by the skilled artisan once provided the teachings disclosed herein.

The compositions of the present disclosure can be administered by parenteral (e.g., intramuscular, intraperitoneal, intravenous, ICV, intracisternal injection or infusion, subcutaneous injection, or implant), oral, by inhalation spray nasal, vaginal, rectal, sublingual, urethral (e.g., urethral suppository) or topical routes of administration (e.g., gel, ointment, cream, aerosol, etc.) and can be formulated in suitable dosage unit formulations containing conventional non-toxic pharmaceutically acceptable carriers, adjuvants, excipients, and vehicles appropriate for each route of administration.

In another aspect, the method further comprises, or consists essentially of, or yet further consists of two or more subsequent cryoablations of the tumor or cancer. In one aspect, the cryoablation is percutaneous cryoablation.

In a further aspect the method and compositions of this disclosure further comprises or consists essentially of, or yet further consists of administration of an effective amount of a checkpoint inhibitor therapy, non-limiting examples of such are known in the art or as disclosed herein. PD-L1 inhibitors include for example Genentech's MPDL3280A (RG7446), anti-PD-LI monoclonal antibody MDX-1105 (BMS-936559) and BMS-935559 from Bristol-Meyer's Squibb, MSB0010718C, and AstraZeneca's MEDI4736. PD-L2 inhibitors include for example GlaxoSmithKline's AMP-224 (Amplimmune), and rHIgM12B7. PD-1 inhibitors include for example, such as anti-mouse PD-1 antibody Clone J43 (Cat #BE0033-2) from BioXcell, anti-mouse PD-1 antibody Clone RMP1-14 (Cat #BE0146) from BioXcell, mouse anti-PD-1 antibody Clone EH12, Merck's MK-3475 anti-mouse PD-1 antibody (Keytruda, pembrolizumab, lambrolizumab), AnaptysBio's anti-PD-1antibody known as ANB011, antibody MDX-1 106 (ONO-4538), Bristol-Myers Squibb's human IgG4 monoclonal antibody nivolumab (OPDIVOR, BMS-936558, MDX1106), AstraZeneca's AMP-514 and AMP-224, and Pidilizumab (CT-011) from CureTech Ltd. CTLA-4 inhibitors include for example Bristol Meyers Squibb's anti-CTLA-4 antibody ipilimumab (also known as YERVOY®, MDX-010, BMS-734016 and MDX-101), anti-CTLA4 antibody clone 9H10 from Millipore, Pfizer's tremelimumab (CP-675,206, ticilimumab), and anti-CTLA4 antibody clone BNI3 from Abeam. LAG3 inhibitors include for example anti-Lag-3 antibody clone eBioC9B7W (C9B7W) from eBioscience, anti-Lag3 antibody LS-B2237 from LifeSpan Biosciences, IMP321 (ImmuFact) from Immutep, anti-Lag3 antibody BMS-986016, and the LAG-3 chimeric antibody A9H12.

The methods can be administered to the subject in need thereof as a first-line, second-line, third-line, fourth-line, or fifth-line therapy. The therapy can be administered to the subject in need thereof to achieve one or more of a reduction in tumor burden, increased time to or lack of tumor recurrence, complete or partial response, or increased overall survival, as compared to a subject who has not received the treatment.

Yet further provided is a kit comprising a composition comprising a VLP (naturally occurring or engineered VLP, e.g. CPMV, with or without a TLR agonist) and instructions for use in the methods as disclosed herein.

### Diagnostic Methods

In some embodiments, one or more of the methods described herein further comprise, or consist essentially of, or yet further consist of, a diagnostic step. In some instances, a sample is first obtained from a subject suspected of having a disease or condition described above or for inducing an immune response in the subject. Exemplary samples include, but are not limited to, cell sample, tissue sample, tumor biopsy, liquid samples such as blood and other liquid samples of biological origin (including, but not limited to, peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, vaginal discharge, sweat, tears, cyst fluid, pleural fluid, peritoneal fluid, pericardial fluid, ascites, lymph, chyme, chyle, bile, interstitial fluid, menses, pus, sebum, vomit, mucosal secretion, stool water, pancreatic juice, lavage fluids from sinus cavities, bronchopulmonary aspirates, blastocyl cavity fluid, or umbilical cord blood. In some instances, the sample is a tumor biopsy. In some cases, the sample is a liquid sample, e.g., a blood sample. In some cases, the sample is a cell-free DNA sample.

Various methods known in the art can be utilized to determine the presence of a disease or condition described herein or to determine whether an immune response has been induced in a subject. Assessment of one or more biomarkers associated with a disease or condition, or for characterizing whether an immune response has been induced, can be performed by any appropriate method. Expression levels or abundance can be determined by direct measurement of expression at the protein or mRNA level, for example by microarray analysis, quantitative PCR analysis, or RNA sequencing analysis. Alternatively, labeled antibody systems may be used to quantify target protein abundance in the cells, followed by immunofluorescence analysis, such as FISH analysis.

### Experimental Materials and Methods

CPMV particle preparation: CPMV particles were propagated in California Blackeye No. 5 cowpea plants and were harvested, purified, and characterized, as previously described.29 In brief, the primary leaves were dusted with abrasive carborundum and mechanically inoculated with 0.05 mg CPMV per leaf. Infected primary leaves and trifoliates were then harvested (10-15 days post inoculation), CPMV and the virus was purified by chloroform-butanol extraction, PEG precipitation, and sucrose gradient ultracentrifugation. CPMV was characterized by SDS-PAGE, size exclusion chromatography using a Superose 6 Increase column and fast liquid protein chromatography, as well as transmission electron microscopy. Additional methods are known in the art to prepare CPMV particles, see e.g. Chan, S. K. et al., Biomimetic Virus-Like Particles as Severe Acute Respiratory Syndrome Coronavirus 2 Diagnostic Tools. ACS Nano 2020, acsnano.0c08430 and Chan, S. K. et al., Virus-Like Particles as Positive Controls for COVID-19 RT-LAMP Diagnostic Assays. Biomacromolecules 2021.

Cell culture: RIL-175 cells (from Dr. Timothy Greten, NCI) were previously isolated from hepatic tumors established in C57BL/6 mice via transfer of p53-/- fetal hepatoblasts transduced with HRasV12.30-32 Cells are maintained in Dulbecco's Modified Eagle's Medium (DMEM; product 10-013-CV-1; Corning) supplemented with 1% (w/v) penicillin-streptomycin solution (product 30-002-CI-1; Corning), 1% (w/v) Minimal Essential Media Non-Essential Amino Acids Solution (product 11140050; ThermoFisher), and 10% (w/v) heat-inactivated fetal bovine serum (16140071; Gibco). Cells are incubated in a humidified 5% CO2 environment and passaged at 1:3 approximately every 2 days.

HCC tumor model: All animal experiments were approved by the UC San Diego Institutional Animal Care and Use Committee. To induce hepatic steatosis, male wildtype C57BL/6 mice (Jackson Laboratories) were fed a nonalcoholic steatohepatitis (NASH)-inducing Western Diet (product D12079; Research Diets, Inc.) beginning at 8-10 weeks old, for at least 4 with subcutaneous tumors. Applicant also observed that tumors tend to regress spontaneously in female mice, so only males were included. For the subcutaneous injections, isoflurane anesthesia was administered by nose cone, the bilateral flanks were shaved, and skin was sanitized with iodine solution.

Tumor cell injection: For the primary tumor slated for treatment, 2.5×10⁵ RIL-175 cells (aggressive murine HCC cell line) suspended in 25 μl of Cultrex Basement Membrane Extract (BME) were injected with a 22G needle into the subcutaneous tissue of the right flank of 42 wild type C57BL/6 mice. For the secondary contralateral tumor, which was to be left untreated, a 1.25×10⁵ RIL-175 cell suspension was injected subcutaneously into the left flank of the same mice at the time of primary tumor injection. The smaller cell inoculation simulates metastasis and ensures that the untreated tumor size does not limit experiment duration. After 10 days of tumor growth, the primary tumors were subsequently treated while the secondary tumors were monitored for an abscopal effect.

Ultrasound-guided, percutaneous cryoablation and intratumoral CPMV injection: Ten days after tumor cell injection, the mice were assessed for engraftment. Tumors were measured with digital calipers, and the length (greatest diameter) and width (diameter orthogonal to length) were recorded. Volume was calculated by the modified ellipsoidal formula: V=½(Length×Width2). Tumors with a volume of at least 85 mm³ were included for analysis.

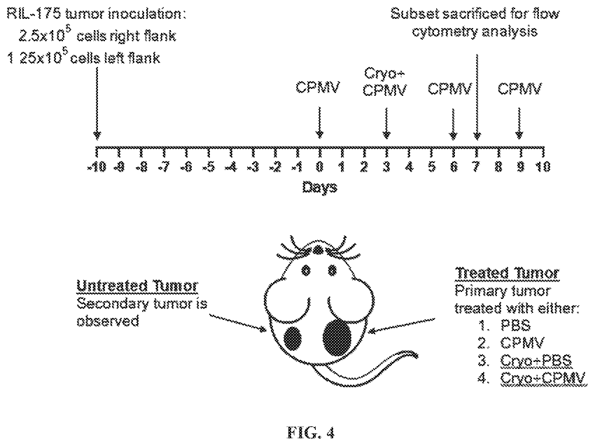

Tumor treatment: The mice were randomized into one of four groups: PBS injected (Control), CPMV-treated only (CPMV), cryoablation only (Cryo), and cryoablation plus CPMV-treated (Cryo+CPMV); n=10-12 per group (day 10 (Group A), CPMV only (Group B), Cryoablation only (Group C), and Combined Cryoablation and CPMV (Group D). On days 10, 13, 16, and 19, the primary tumor of each mouse in the two CPMV groups was directly injected with either 20 μl of PBS (Groups A and C) or 20 μl CPMV (5 μg/μL using a 26G needle. Control and Cryo mice received intratumoral PBS injections. On day 13, Groups B and D). On day 3, the primary tumors in the two Cryo groups of Groups C and D were treated with cryoablation using a custom 3-cm, 17G IceSeed cryoprobe (Galil Medical Cryoablation System, Probe (Boston Scientific) inserted into the center of the tumor under ultrasound guidance with a 71 MHz US transducer (Vevo MD Ultra High Frequency ultrasound system; VisualSonics, Fujifilm) and subjected to two and 2 cycles of 1-minute active freezes and 1-minute active thaws at 25% power to generate a 7-mm ablation zone. FIG. 4 summarizes the study design and treatment schedule.

Data collection: Tumor growth was measured with digital calipers. Tail bleed serum samples were obtained at days 0, 3 and 6 to assess chemokine/cytokine concentrations. Two mice from each group were sacrificed at 1 week post-treatment and tumors were harvested for flow cytometry.

Mice were weighed every 3 days, beginning before tumor cell injection and continuing until sacrifice. Animals were sacrificed prior to the established endpoints if tumor volume reached 1 cm³, weight decreased by 15% compared to baseline, or the animal appeared moribund.

Chemokine/Cytokine Analysis: Tail bleed samples were collected in EDTA tubes at days 0, 3 and 6, and samples were centrifuged at 6000 RPM for 6 minutes at 4° C. The supernatant was stored at-80° C. prior to analysis using an electrochemiluminescence-based multiplex immunoassay (Meso Scale Diagnostics, Rockville, MD). IFN-γ, IL-1B, IL-2, IL-4, IL-5, IL-10, IL-12p70, IL-13, CXCLI and TNF-α were measured with the U-plex TH1/TH2Combo kit (product K15071K), according to the manufacturer's instructions.

Flow Cytometry: At 7 days post-treatment initiation, 2 mice from each group were sacrificed for flow cytometry analysis. Following isoflurane anesthesia and cervical dislocation, the spleen and tumors were collected. Single cell suspensions were obtained through digestion with RPMI/Enzyme solution (Tumor dissociation kit, Miltenyi Biotec) followed by mechanical dissociation using gentleMACS tissue dissociator (Miltenyi Biotec). The single cell suspensions (diluted to 1.0×107 cells/mL) were incubated for 20 min at 4° C. with anti-mouse CD16/32 (Biolegend) to block the Fc receptors and stained with the following anti-mouse antibodies (1:500 dilution, BioLegend) for 1 hour at 4° C. in the dark: CD45 Peg CP/Cy5.5 (30-F11), CD3 AF488 (17A2), CD4 Brilliant violet 605 (RM4-4), CD8 Brilliant violet 785 (53-6.7), CD137 PE (17B5), I-A/I-E PE/Cy7 (M5/114.15.2), NK1.1 APC/Cy7 (S17016D), CD134 APC (OX-86), CD279 APC-R700 (29F.1A12), Ly-6G PE-eFluor 610 (1A8-Ly6g), CD11b Super Bright 645 (M1/70), and CD11c Super Bright 780 (N418). Stained cells were fixed with BD-stabilizing fixative and resuspended in 200 μL of FACS buffer (2% (v/v) fetal bovine serum in PBS) and analyzed on a Beckman-Coulter Cytoflex machine. Data was analyzed with CytExpert (Beckman) and FlowJo (FlowJo LLC). hematoxylin and eosin (HE) staining, and immunohistochemistry (IHC). Tissues were probed with primary antibodies targeted against murine CD4 (Abcam, Cat #ab183685) and CD8 (ThermoFisher, Cat #14-0195-82). Slides were scanned on a Nanozoomer digital slide scanner (Hamamatsu) at the UCSD Microscopy Core. Positive cell counts were quantitated using the Fiji image processing package33 at 10× (for overall) and 20× (for central vs. peripheral) magnification and divided by the total quantitated area to determine positive cell count/mm².

History: At 7 days post-treatment initiation, tumors from a subset of mice from each group (N=1-2) were harvested by blunt dissection, followed by sectioning and fixation in 4% (v/v) formaldehyde in PBS. All histological samples were submitted to the Moores Cancer Center Biorepository and Tissue Technology shared resource for paraffin embedding, thin sectioning,

Data Analysis and Statistical Methods: One-way ANOVA was performed in Prism (GraphPad) for all statistical comparisons among treatment groups. Flow cytometry data was analyzed in FlowJo (Treestar) and imported into Prism for statistical analysis. A p value of <0.05 was used as the threshold for statistical significance.

### Results

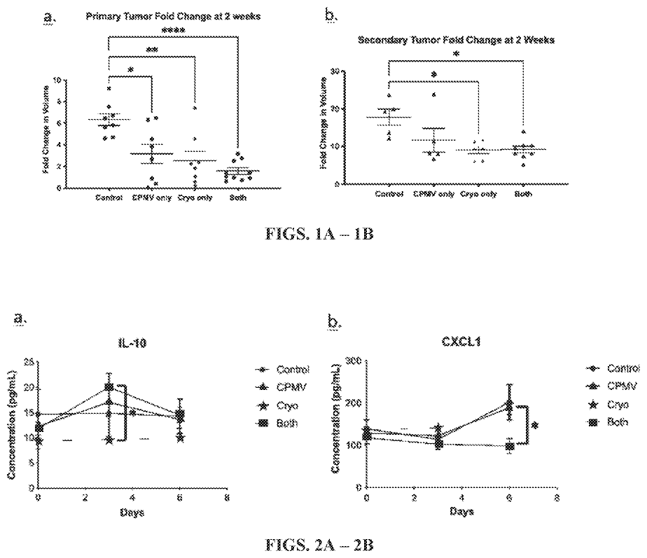

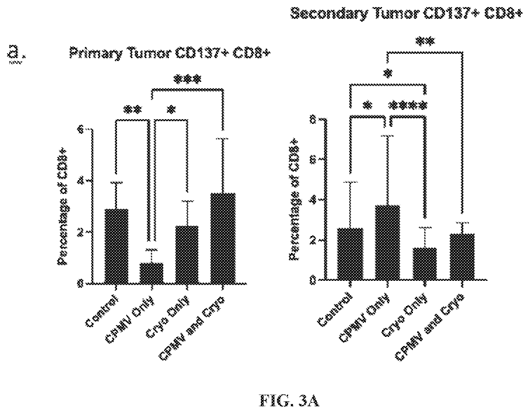

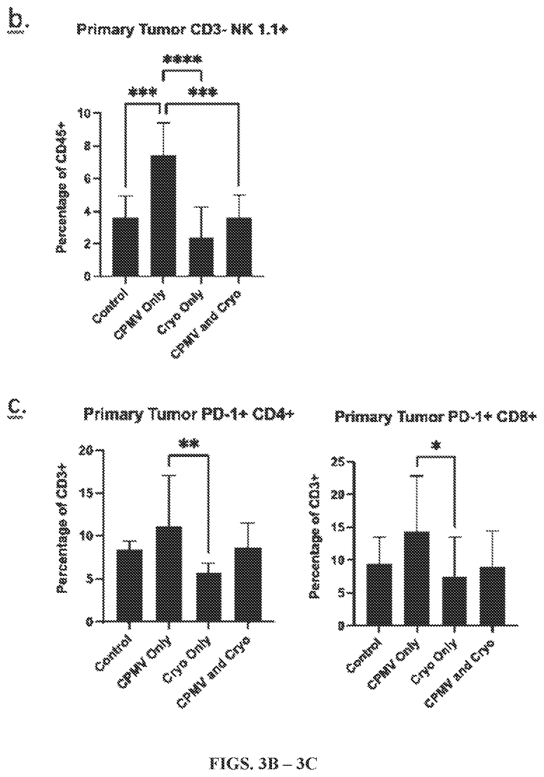

At 2 weeks post treatment, the combined treatment Group D demonstrated the smallest magnitude of relative growth increase as compared to control Group A (1.6±0.9 vs. 6.3±0.5, p<0.0001) (FIG. 1A). The secondary tumor of the cryoablation only Group C and combined treatment Group D showed similar magnitudes of reduced growth rates as compared to control Group A (FIG. 1B). Analysis of systemic cytokine/chemokine concentrations revealed a transient increase of IL-10 in the combined treatment Group D compared to the cryoablation only Group C (20.0±2.7 pg/mL vs. 9.6±1.0 pg/mL; p=0.03). However, this difference diminished at 6 days (FIG. 2A). There was also a significantly lower concentration of CXCL1 in the combined treatment Group D as compared with the CPMV Group B at 6 days (98.2±17.6 pg/mL vs. 189.0±16.6 pg/mL; p=0.01) (FIG. 2B). Flow cytometry analysis revealed an enrichment of activated CD8+/CD137+ T cells in the primary tumor compared with the CPMV only group (3.6% vs 0.8%, p=0.0002). Paradoxically, in the secondary tumor, the CPMV only Group B had the highest percentage of activated CD8+/CD137+ T cells (FIG. 3A). The CPMV only Group B also had the highest percentage of NK cells in the primary tumor (FIG. 3B). Finally, there was increased PD-1 expression on both CD4+ and CD8+ T cells in the CPMV-treated tumors of Group B (FIG. 3C).

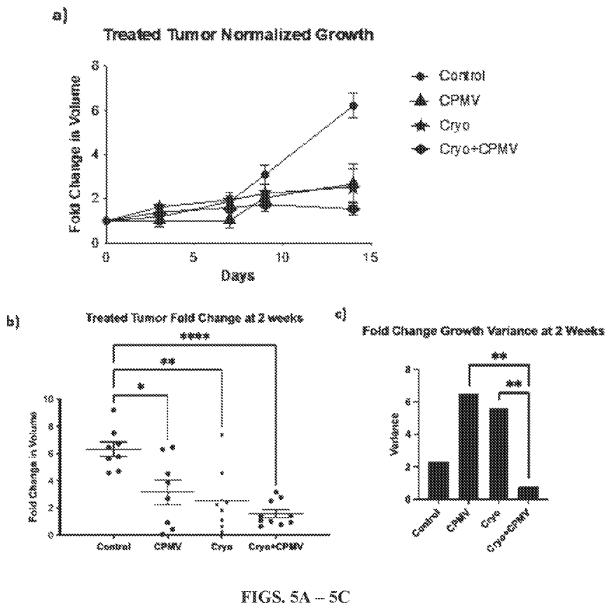

Tumor Growth: Tumor size was monitored over time, and all three treatment groups demonstrated slowed tumor growth compared to Control, with the Cryo+CPMV group demonstrating the greatest growth retardation (FIG. 5A). At sacrifice, it was confirmed that Cryo+CPMV resulted in the greatest reduction of tumor growth vs. Control (1.6±0.9 vs. 6.3±0.5, p<0.0001) (FIG. 5B). Cryo+CPMV showed a trend toward more tumor growth inhibition than Cryo or CPMV alone; however, the differences were not statistically significant, as the response to the individual treatments showed large variation. Analysis of the variance in treatment response indicated that Cryo+CPMV treatment had significantly lower variance, thus yielding a more consistent response than either treatment alone (F test of Cryo+CPMV vs. CPMV: 0.82 vs 6.52, p=0.006; and Cryo+CPMV vs. Cryo: 0.82 vs. 5.65, p=0.01) (FIG. 5C).

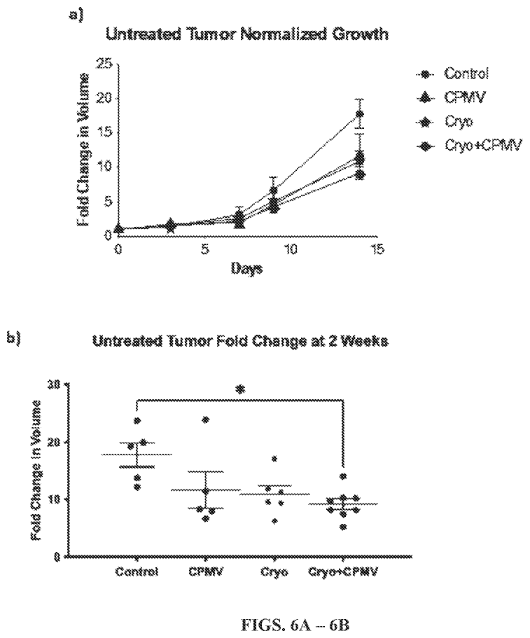

The growth of the contralateral, untreated tumor was also monitored over time. In the three treatment groups, the untreated tumor showed a trend towards decreased growth over time, but the Cryo+CPMV group was the only one that reached significance compared with the Control group (9.2±0.9 vs 17.8±2.1, p=0.01) (FIG. 6).

Cytokine/Chemokine Profiles: A panel of chemokines and cytokines were measured in plasma samples from the mice (Table 2). Statistically significant changes were only observed for IL-10 and CXCL1. In the Cryo+CPMV group, IL-10 was significantly increased at day 3 post treatment compared to the Cryo group (20.0±2.7 pg/mL vs. 9.6±1.0 pg/mL; p=0.03) (FIG. 6A). However, this difference did not persist at day 6. IL-10 tended to be increased CPMV-treated mice, but it did not reach statistical significance. In contrast, CXCL1 was significantly reduced at day 6 in Cryo+CPMV tumors compared with CPMV tumors (98.2±17.6 pg/mL vs. 189.0±16.6 pg/mL; p=0.01), a level that was essentially unchanged from day 3 (FIG. 6B). While Control and Cryo groups had similar concentrations of CXCL1 compared with CPMV, the differences of these groups vs Cryo+CPMV did not reach statistical significance.

TABLE 2
Fold change in mean concentration of serum chemokine/cytokine
levels. Significant differences were found for IL-10 and
CXCL1 concentrations, which are highlighted in bold.

| | Fold Change in Mean Concentration | | | | | | | |
|---|---|---|---|---|---|---|---|---|
| | Control | | CPMV | | Cryo | | Cryo + CPMV | |
| | Day | Day | Day | Day | Day | Day | Day | Day |
| Cytokine/Chemokine | 3 | 6 | 3 | 6 | 3 | 6 | 3 | 6 |
| IFN-γ | 0.96 | 0.74 | 0.90 | 1.04 | 1.05 | 0.87 | 1.22 | 1.17 |
| IL-1 β | 7.69 | 9.00 | 4.93 | 5.05 | 4.85 | 7.84 | 5.76 | 4.82 |
| IL-2 | 1.01 | 0.94 | 1.00 | 1.07 | 1.00 | 1.03 | 0.87 | 1.10 |
| IL-4 | 0.99 | 0.95 | 1.08 | 1.02 | 1.52 | 1.08 | 1.54 | 0.45 |
| IL-5 | 1.00 | 0.97 | 0.90 | 0.83 | 0.86 | 1.08 | 1.05 | 1.05 |
| IL-10 | 1.01 | 0.96 | 1.34 | 0.80 | 1.03 | 1.06 | 1.7 | 1.23 |
| IL-12p70 | 0.32 | 0.38 | 0.99 | 0.53 | 0.61 | 0.53 | 1.01 | 0.62 |
| IL-13 | 1.02 | 0.90 | 0.88 | 0.17 | 1.42 | 1.37 | 0.90 | 0.77 |
| CXCL1 | 0.82 | 1.45 | 0.95 | 1.46 | 1.03 | 1.43 | 0.87 | 0.83 |
| TNF- α | 0.99 | 1.34 | 1.22 | 1.49 | 1.32 | 1.16 | 1.13 | 1.23 |

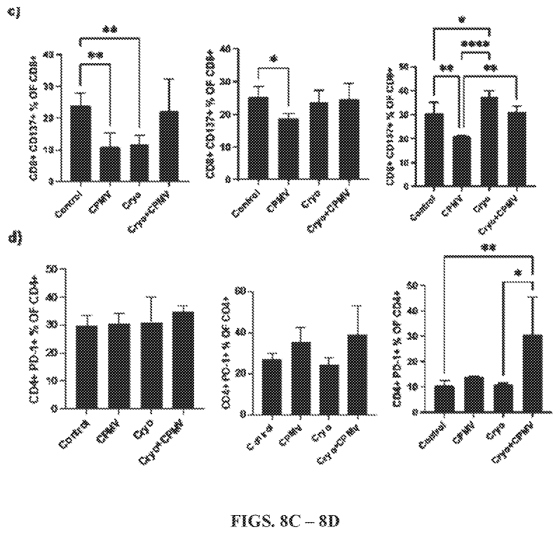

Flow Cytometry: Applicant profiled immune cell infiltration into the treated and untreated tumors and in the spleen. No significant differences were seen in total CD4+ or CD8+ T-cell populations in treated tumors and spleens. CD4+ T-cells were enriched in the contralateral, untreated tumors of mice treated with Cryo+CPMV compared with Cryo (12.9% vs 8.6%, p=0.03) (FIG. 8A). CD8+ T-cells were enriched both in untreated tumors of mice treated with CPMV compared with Control (8.6% vs 3.6%, p=0.005) as well as untreated tumors of mice treated with Cryo+CPMV compared with Control (7.8% vs 3.6%, p=0.03) (FIG. 8B). CD8+/CD137+ T cells are CD8+ T cells activated in the presence of antigens. In the treated tumors, CD8+/CD137+ T cells were decreased in the CPMV group (10.9% vs. 24.0%, p=0.01) and Cryo group (12.0% vs 24.0%, p=0.01) compared with Control. However, Cryo+CPMV appeared to reverse this inhibition (FIG. 8C). Similarly, in the untreated tumors, activated CD8+/CD137+ T cells were decreased in the CPMV group compared with Control (18.8% vs. 25.4%, p=0.04), whereas Cryo+CPMV was not statistically significant compared to control. In the spleen, activated CD8+/CD137+ T cells were reduced in the CPMV group compared with Control (21.0% vs. 30.7%, p=0.003), but the Cryo group demonstrated higher activated CD8+/CD137+ T cells compared with Control (37.8% vs. 30.7%, p=0.03) and CPMV (37.8% vs. 21.0%, p<0.0001). Cryo+CPMV normalized the number of activated CD8+/CD137+ T cells compared to Control. Finally, PD-1 expression on CD4+ and CD8+ T cells was not changed in the treated tumors. PD-1 expression on CD8+ T cells was increased in untreated tumors in the Cryo+CPMV group compared with Cryo (72.5% vs. 51.8%, p=0.03). In spleens, the Cryo+CPMV group had more PD-1+/CD4+ and PD-1+/CD8+ T cells compared with both Control (CD4+ 30.9% vs. 10.6%, p=0.007; CD8+13.7% vs. 3.6%, p=0.008) and Cryo (CD4+ 30.9% vs. 11.2%, p=0.03; CD8+9 13.7% vs. 4.0%, p=0.04) (FIG. 8D and FIG. 8E). For NK cells, the treated tumors did not show a change, but untreated tumors in the CPMV group showed increased NK cells compared with both Control (18.3% vs. 8.6%, p=0.002) and Cryo (18.3% vs. 10.5%, p=0.009) (FIG. 8F). Cryo+CPMV also increased NK cells in the untreated tumor compared with Control (15.4% vs. 8.6%, p=0.04). In spleens, Cryo+CPMV increased NK cells compared with both Control (6.3% vs. 3.7%, p=0.01) and CPMV (6.3% vs. 2.4%, p=0.003).

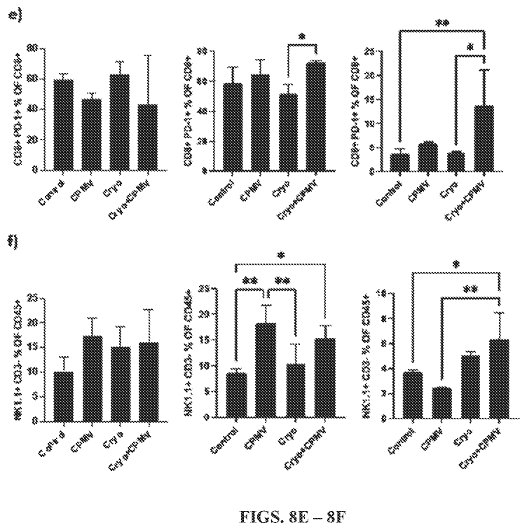

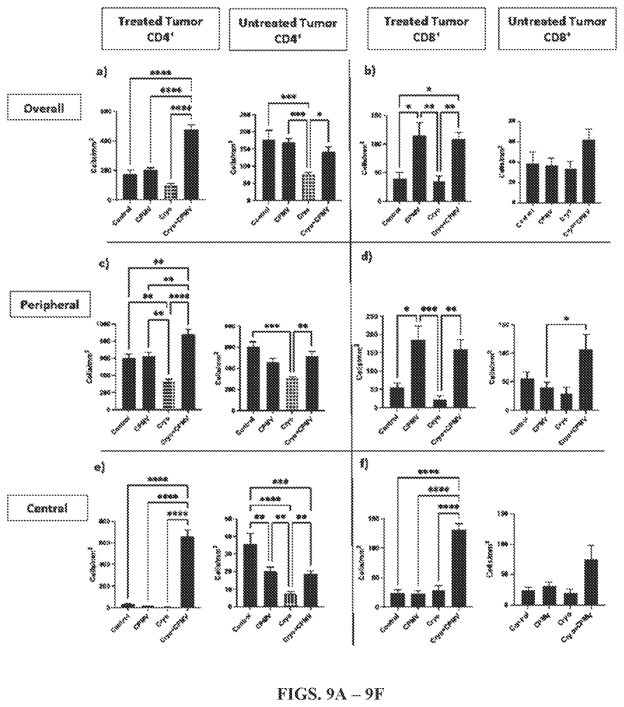

Histology: Treated and untreated tumor sections were stained for CD4+ and CD8+ lymphocytes, and positive cells were separately quantified based on overall, peripheral, and central tumor distributions. Overall CD4+ lymphocyte concentration was increased in Cryo+CPMV treated tumors (479/mm2) compared to all other groups (p<0.0001). In the untreated tumor, CD4+ lymphocyte concentration was decreased in the Cryo group (77/mm2) compared to all other groups (FIG. 9A). Overall CD8+ cell concentration was higher for CPMV (114/mm2) and Cryo+CPMV (109/mm2) as compared to Control (39/mm2) and Cryo (36/mm2). In the untreated tumor, CD8+ cells trended higher in Cryo+CPMV as compared to all other groups, but it did not reach statistical significance (FIG. 9B). CD4+ count in the treated tumor periphery was highest inCryo+CPMV (878/mm2) and lowest in Cryo (332/mm2), as compared to all other groups. CD4+ count in the untreated tumor periphery was higher in both Control (602/mm2) and Cryo+CPMV (516/mm2) as compared to Cryo (307/mm2) (FIG. 9C). Peripheral infiltration of CD8+ cells was increased in both CPMV (184/mm2) and Cryo+CPMV (159/mm2) as compared to Control (55/mm2) and Cryo (24/mm2). In the untreated tumor, peripheral CD8+ cells were higher in Cryo+CPMV (107/mm2) as compared to CPMV (39/mm2) (FIG. 9D). In the center of the tumor treated with Cryo+CPMV, CD4+ cells (664/mm2) and CD8+ cells (131/mm2) were dramatically increased compared to all other groups (p<0.0001) (FIGS. 6e and f). In the untreated tumor, central CD4+ count was lowest in the Cryo group (8/mm2). There was a trend for increased CD8+ infiltration centrally in Cryo+CPMV compared to all other groups, but this did not reach statistical significance. FIG. 10 shows representative examples of CD4+ staining. Notably, tumor fat content was markedly increased in Cryo+CPMV treated tumors (FIG. 10A) compared to all other groups.

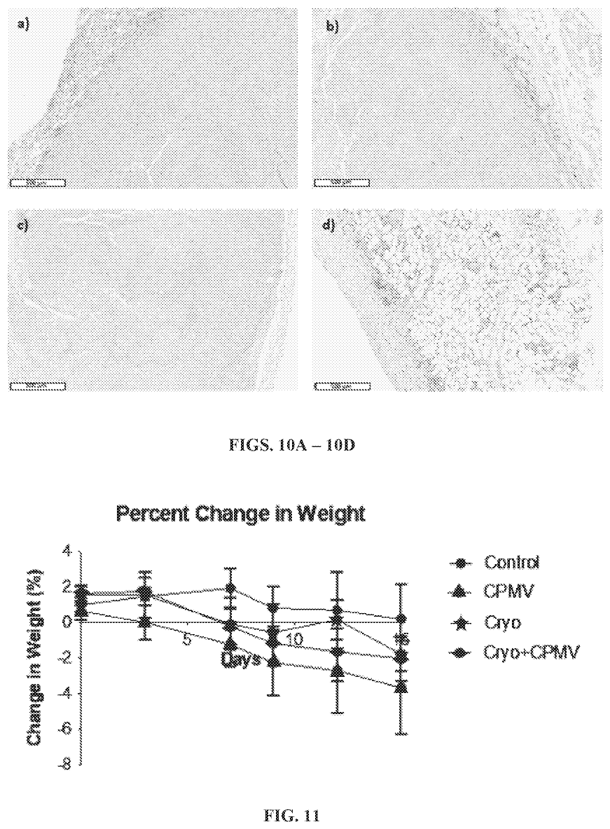

Treatment toxicity and weight loss: CPMV-treated animals tended to have lower weight than Control and Cryo animals, but these differences did not reach statistical significance (FIG. 11). No animals reached the 15% weight loss or 1 cm3 tumor volume threshold for euthanasia or appeared moribund during the 2-week_experimental period.

### Discussion

Multimodal ISV therapy consisting of cryoablation combined with the immunostimulant CPMV inhibited the growth of both the treated and contralateral, untreated HCC tumors and promoted an immunogenic tumor microenvironment (TME).

The reported data support an immune-mediated effect of cryoablation plus CPMV on tumor growth. The combination of cryoablation and intratumoral CPMV promoted lymphocyte infiltration into the center of the tumors, compatible with a shift from an immunologically “cold” to a “hot” tumor. Multiparametric flow cytometry and IHC highlighted important differences in both native and adaptive immune cell populations among treatment groups in the untreated tumors and spleens, as well as differences in geographical distribution. Previous work with CPMV in other tumor models also demonstrated marked increases in CD4+, CD8+ and NK cells in treated tumors.^{25.37} Wang et al. demonstrated that CPMV protected subjects against ovarian cancer rechallenge and elicited tumor-specific CD4+ and CD8+ immune memory cells.²² The potential for clinical translation of this multimodal ISV therapy for patients with intermediate or advanced HCC (BCLC B or C) is promising, but earlier stage patients could benefit, as well. Combining cryoablation with an immunostimulant could ensure more complete responses to ablation and reduce recurrence through immune elimination of residual cancer cells. This could impact mortality, since sublethal thermal stress stimulates residual cancer cells to proliferate and grow, which can drive more aggressive recurrence.³⁴⁻³⁶ It is also favorable for clinical applications that the multimodal ISV therapy yielded more consistent tumor growth retardation than cryoablation or CPMV alone.

The immunological impact of cryoablation may depend on factors such as tumor type, volume of tumor frozen, the freeze-thaw protocol, and whether appropriate immunoadjuvants or costimulatory signaling markers are presented alongside TAA. Although cryoablation might be expected to promote immunostimulation through the release of TAA and pro-inflammatory cytokines, this study and others have demonstrated immunosuppressive effects.³⁸ Cryoablation alone appeared to elicit an immunosuppressive effect in this HCC model that was reversed with the addition of CPMV. To explore this further, PD-1 expression was analyzed, since RIL-175 hepatoma cells are known to express PD-L1.³⁹ PD-1 expression on both CD4+ and CD8+ T cells was significantly increased in the spleen, which suggests that a component of immune exhaustion still occurs with Cryo+CPMV. Thus, the addition of a PD-1 inhibitor can be used to further enhance the efficacy of this combined treatment. Previous work with CPMV in multiple other tumor models found a strong upregulation of CPIs and improved outcomes with the addition of PD-1 inhibitors.³⁷

On histology, Cryo+CPMV induced a marked increase in centrally-penetrating tumor-infiltrating lymphocytes (TILs), which characterizes immunogenic (or “hot”) tumors. Impaired trafficking of TILs is an immunoevasive strategy by HCC.⁴⁰ The presence of TILs is strongly associated with an immune-mediated anti-tumor response, as well as the abscopal effect.^{41.42} The treatment effect of Cryo+CPMV may therefore be related to enhanced TIL recruitment and/or improved tumor permeability to TILs. Histologic specimens also demonstrated a marked increase in the fat content of tumors treated with Cryo+CPMV.

Significant differences were found in IL-10 and CXCL1 concentrations among treatment groups. Although IL-10 is generally considered anti-inflammatory and involved in the suppression or resolution of infection, it may also have pro-inflammatory activity.⁴³ IL-10 was increased in the Cryo+CPMV group after 3 days. Without being bound by theory, this may reflect a systemic effect to curb the early inflammation occurring within the TME or could reflect a pro-inflammatory effect. CXCLI is a chemokine that attracts neutrophils and other immune cells during inflammation and promotes angiogenesis and tumor growth in liver cancer⁴⁴. CXCL1 also enhances immunotolerance by recruiting immunosuppressive myeloid-derived stem cells.⁴⁵ CXCLI was significantly decreased at 6 days in the Cryo+CPMV group compared to all other groups, perhaps resulting in an anti-tumor effect by limiting angiogenesis and relieving immunosuppression.

Combining cryoablation with intratumoral CPMV produced the greatest growth reduction in the primary tumor and demonstrated a modest abscopal effect in the contralateral, untreated tumor, in this model of aggressive HCC. This growth reduction is associated with markedly increased tumor core-infiltrating CD4+ and CD8+ T cells and NK cells and reduced expression of CXCL1. Increased expression of PD-1 on CD4+ and CD8+ T cells suggests a potential role for the addition of a PD-1 inhibitor to enhance the efficacy of cryoablation plus CPMV.

Thus, in sum, this work shows that combining intratumoral CPMV with cryoablation produced the greatest reduction in primary tumor growth. Without being bound by theory, this tumor growth retardation may be due to an enrichment of CD8+ T cells that are activated by the presence of TAAs, stimulation of NK cells by CPMV, and reduced expression of CXCL1. Increased PD-1 expression was seen CPMV-treated tumors and therefore the addition of PD-1 inhibitor therapy can further enhance the efficacy of CPMV plus cryoablation.

### Equivalents

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### REFERENCES

1. Bray F, Ferlay J, Soerjomataram I, et al. Global cancer statistics 2018: GLOBOCAN estimates of incidence and mortality worldwide for 36 cancers in 185 countries. CA Cancer J Clin. 2018; 68:394-424.
2. El-Serag H B, Kanwal F. Epidemiology of hepatocellular carcinoma in the United States: Where are we? Where do we go? Hepatology. 2014; 60:1767-1775.
3. Blank C U. The perspective of immunotherapy: new molecules and new mechanisms of action in immune modulation. Curr Opin Oncol. 2014; 26:204-214.
4. Pang Y-L, Zhang H-G, Peng J-R, et al. The immunosuppressive tumor microenvironment in hepatocellular carcinoma. Cancer Immunol Immunother. 2009; 58:877-886.
5. Yau T, Park J W, Finn R S, et al. LBA38_PRCheckMate 459: A randomized, multi-center phase III study of nivolumab (NIVO) vs sorafenib (SOR) as first-line (1L) treatment in patients (pts) with advanced hepatocellular carcinoma (aHCC). Ann Oncol. 2019; 30
6. Finn R S, Ryoo B-Y, Merle P, et al. Results of KEYNOTE-240: phase 3 study of pembrolizumab (Pembro) vs best supportive care (BSC) for second line therapy in advanced hepatocellular carcinoma (HCC). J Clin Oncol. 2019; 37:4004.
7. Finn R S, Qin S, Ikeda M, et al. Atezolizumab plus bevacizumab in unresectable hepatocellular carcinoma. N Engl J Med. 2020; 382:1894-1905.
8. Jansen M C, van Hillegersberg R, Schoots IG, et al. Cryoablation induces greater inflammatory and coagulative responses than radiofrequency ablation or laser induced thermotherapy in a rat liver model. Surgery. 2010; 147:686-695.
9. Mehta A, Oklu R, Sheth R A. Thermal Ablative Therapies and Immune Checkpoint Modulation: Can Locoregional Approaches Effect a Systemic Response? Gastroenterol Res Pract. 2016; 2016:9251375.
10. Ahmad F, Gravante G, Bhardwaj N, et al. Changes in interleukin-1β and 6 after hepatic microwave tissue ablation compared with radiofrequency, cryotherapy and surgical resections. Am J Surg. 2010; 200:500-506.
11. Erinjeri J P, Thomas C T, Samoilia A, et al. Image-guided thermal ablation of tumors increases the plasma level of interleukin-6 and interleukin-10. J Vasc Interv Radiol. 2013; 24:1105-1112.
12. Shao Q, O'Flanagan S, Lam T, et al. Engineering T cell response to cancer antigens by choice of focal therapeutic conditions. Int J Hyperthermia. 2019; 36:130-138.
13. Kumar A V, Patterson S G, Plaza M J. Abscopal Effect following Cryoablation of Breast Cancer. J Vasc Interv Radiol. 2019; 30:466-469.
14. Uhlschmid G, Kolb E, Largiadèr F. Cryosurgery of pulmonary metastases. Cryobiology. 1979; 16:171-178.
15. Joosten J J, Muijen G N, Wobbes T, et al. In vivo destruction of tumor tissue by cryoablation can induce inhibition of secondary tumor growth: an experimental study. Cryobiology. 2001; 42:49-58.
16. Pillarisetty V G, Shah A B, Miller G, et al. Liver dendritic cells are less immunogenic than spleen dendritic cells because of differences in subtype composition. J Immunol. 2004; 172:1009-1017.
17. Bogdanos D P, Gao B, Gershwin M E. Liver immunology. Compr Physiol. 2013; 3:567-598.
18. Murray A A, Wang C, Fiering S, et al. In Situ Vaccination with Cowpea vs Tobacco Mosaic Virus against Melanoma. Mol Pharm. 2018; 15:3700-3716.
19. Lizotte P H, Wen A M, Sheen M R, et al. In situ vaccination with cowpea mosaic virus nanoparticles suppresses metastatic cancer. Nat Nanotechnol. 2016; 11:295-303.
20. Kerstetter-Fogle A, Shukla S, Wang C, et al. Plant Virus-Like Particle In Situ Vaccine for Intracranial Glioma Immunotherapy. Cancers (Basel). 2019; 11
21. Wang C, Steinmetz N F. CD47 Blockade and Cowpea Mosaic Virus Nanoparticle In Situ Vaccination Triggers Phagocytosis and Tumor Killing. Adv Healthc Mater. 2019; 8: e1801288.
22. Wang C, Fiering S N, Steinmetz N F. Cowpea Mosaic Virus Promotes Anti-Tumor Activity and Immune Memory in a Mouse Ovarian Tumor Model. Adv Ther. 2019; 2:1900003.
23 Shukla S, Jandzinski M, Wang C, et al. A Viral Nanoparticle Cancer Vaccine Delays Tumor Progression and Prolongs Survival in a HER2+ Tumor Mouse Model. Adv Ther. 2019; 2:1800139.
24. Liu L, Chen H, Wang M, et al. Combination therapy of sorafenib and TACE for unresectable HCC: A systematic review and meta-analysis. PLOS One. 2014; 9:1-8.
25. Patel R, Czapar A E, Fiering S, et al. Radiation Therapy Combined with Cowpea Mosaic Virus Nanoparticle in Situ Vaccination Initiates Immune-Mediated Tumor Regression. ACS omega. 2018; 3:3702-3707.
26. Alonso-Miguel D, Valdivia G, Guerrera D, et al. Neoadjuvant in situ vaccination with cowpea mosaic virus as a novel therapy against canine inflammatory mammary cancer. J Immunother cancer. 2022; 10
27. Hoopes P J, Wagner R J, Duval K, et al. Treatment of Canine Oral Melanoma with Nanotechnology-Based Immunotherapy and Radiation. Mol Pharm. 2018; 15:3717-3722.
28. Koellhoffer E C, Steinmetz N F. Cowpea Mosaic Virus and Natural Killer Cell Agonism for In Situ Cancer Vaccination. Nano Lett. 2022; 22:5348-5356.
29. Leong H S, Steinmetz N F, Ablack A, et al. Intravital imaging of embryonic and tumor neovasculature using viral nanoparticles. Nat Protoc. 2010; 5:1406-1417.
30. Kapanadze T, Gamrekelashvili J, Ma C, et al. Regulation of accumulation and function of myeloid derived suppressor cells in different murine models of hepatocellular carcinoma. J Hepatol. 2013; 59:1007-1013.
31. Zender L, Xue W, Cordón-Cardo C, et al. Generation and analysis of genetically defined liver carcinomas derived from bipotential liver progenitors. Cold Spring Harb Symp Quant Biol. 2005; 70:251-261.
32. Brown Z J, Heinrich B, Greten T F. Establishment of Orthotopic Liver Tumors by Surgical Intrahepatic Tumor Injection in Mice with Underlying Non-Alcoholic Fatty Liver Disease. Methods Protoc. 2018; 1
33. Schindelin J, Arganda-Carreras I, Frise E, et al. Fiji: an open-source platform for biological-image analysis. Nat Methods. 2012; 9:676-682.
34. Huo Y, Chen W S, Lee J, et al. Stress Conditions Induced by Locoregional Therapies Stimulate Enrichment and Proliferation of Liver Cancer Stem Cells. JVasc Interv Radiol. 2019
35. Kroeze S G C, van Melick H H E, Nijkamp M W, et al. Incomplete thermal ablation stimulates proliferation of residual renal carcinoma cells in a translational murine model. BJU Int. 2012; 110: E281-6.
36. Yang Y, Wang C, Lu Y, et al. Outcomes of ultrasound-guided percutaneous argon-helium cryoablation of hepatocellular carcinoma. J Hepatobiliary Pancreat Sci. 2012; 19:674-684.
37. Wang C, Steinmetz N F. A Combination of Cowpea Mosaic Virus and Immune Checkpoint Therapy Synergistically Improves Therapeutic Efficacy in Three Tumor Models. Adv Funct Mater. 2020; 30
38. Sabel M S. Cryo-immunology: a review of the literature and proposed mechanisms for stimulatory versus suppressive immune responses. Cryobiology. 2009; 58:1-11.
39. Shigeta K, Datta M, Hato T, et al. Dual Programmed Death Receptor-1 and Vascular Endothelial Growth Factor Receptor-2 Blockade Promotes Vascular Normalization and Enhances Antitumor Immune Responses in Hepatocellular Carcinoma. Hepatology. 2020; 71:1247-1261.
40. Hiraoka N. Tumor-infiltrating lymphocytes and hepatocellular carcinoma: molecular biology. Int J Clin Oncol. 2010; 15:544-551.
41. Khan S Y, Melkus M W, Rasha F, et al. Tumor-Infiltrating Lymphocytes (TILs) as a Biomarker of Abscopal Effect of Cryoablation in Breast Cancer: A Pilot Study. Ann Surg Oncol. 2022; 29:2914-2925.
42. Ding W, Xu X, Qian Y, et al. Prognostic value of tumor-infiltrating lymphocytes in hepatocellular carcinoma: A meta-analysis. Medicine (Baltimore). 2018; 97: e13301.
43. Lu L, Zhang H, Dauphars D J, et al. A Potential Role of Interleukin 10 in COVID-19 Pathogenesis. Trends Immunol. 2021; 42:3-5.
44. Cao Z, Fu B, Deng B, et al. Overexpression of Chemokine (C-X-C) ligand 1 (CXCL1) associated with tumor progression and poor prognosis in hepatocellular carcinoma. Cancer Cell Int. 2014; 14:86.
45. Susek K H, Karvouni M, Alici E, et al. The Role of CXC Chemokine Receptors 1-4 on Immune Cells in the Tumor Microenvironment. Front Immunol. 2018; 9:2159.

1. A method of one or more of: treating or inhibiting the growth of a tumor or cancer in a subject in need thereof comprising administration of an effective amount of a naturally occurring plant virus or an engineered virus like particle (“VLP”) and subsequent ablation of the tumor or cancer, optionally wherein the cancer is localized or metastatic and optionally wherein the naturally occurring plant virus or the engineered VLP is administered in a composition, optionally wherein the administration is systemic or local to the tumor or cancer in the subject, and optionally wherein the ablation of the tumor or cancer comprises a method selected from one or more of cryoablation, irreversible electroporation, radiofrequency ablation, or microwave ablation.

2-3. (Canceled)

4. The method of claim 1, wherein the naturally occurring plant virus or the engineered VLP further comprises at least one toll-like receptor (“TLR”) agonist, optionally a TLR 2, 3, 4, 7, and/or 9 agonist.

5. The method of claim 4, wherein the TLR agonist is packaged within the naturally occurring plant virus or the engineered VLP.

6. The method of claim 4, wherein the naturally occurring plant virus or the engineered VLP binds and activates the TLR.

7. The method of claim 1, wherein the plant virus or the engineered VLP comprises cowpea mosaic virus (CPMV), optionally wherein the CPMV is a naturally occurring CPMV.

8. (canceled)

9. The method of claim 1, wherein the tumor or cancer comprises a solid tumor, optionally a cancer derived from the oropharynx, lung, thyroid, esophagus, stomach, liver, pancreas, colon, rectum, kidney, bladder, adrenal gland, breast, uterus, cervix, ovaries, prostate, testes, mesothelium, peritoneum, skin, brain, spinal cord, bone, or lymph nodes, optionally wherein the solid tumor is selected from the group of a sarcoma, a carcinoma, or a lymphoma, and optionally wherein the solid tumor is localized or metastatic, and optionally wherein the cancer is selected from hepatocellular carcinoma, non-small cell lung cancer, small cell lung cancer, or melanoma.

10. The method of claim 1, wherein the tumor or cancer is a cancer of one or more of: the skin, central nervous system, head and neck, breast, thorax, digestive system, genitourinary system, pelvic organs, or lymphatic system.

11-12. (canceled)

13. The method of claim 1, wherein the subject is an animal or a human.

14. The method of claim 1, wherein a plurality of naturally occurring or engineered VLPs are administered to the subject, that may be the same or different from each other.

15. The method of claim 4, wherein the TLR agonists are the same or different from each other.

16. The method of claim 1, further comprising two or more subsequent ablations of the tumor or cancer, that optionally may comprise the same or different method of ablation.

17. The method of claim 1, further comprising administration of an effective amount of a checkpoint inhibitor therapy to the subject, optionally wherein the checkpoint inhibitor therapy comprises an anti-PD-1 therapy.

18. (canceled)

19. The method of claim 1, wherein the method is administered as a first-line, second-line, third-line, fourth-line, or fifth-line therapy.

20. The method of claim 1, wherein the treatment provides to the subject one or more of a reduction in tumor burden, increased time to or lack of tumor recurrence, complete or partial response, longer progression-free survival, longer disease-free survival, a better objective response rate, lowered toxicity or side effects, or increased overall survival, as compared to a subject who has not received the treatment.

21. A composition comprising a naturally occurring plant virus or engineered virus like particle (“VLP”) wherein the naturally occurring plant virus or VLP further comprises a toll-like receptor (“TLR”) agonist, optionally a TLR 2, 3, 4, 7 and/or 9 agonist, and optionally a carrier, and further optionally a detectable label, optionally wherein the TLR agonist is packaged within the naturally occurring plant virus or the engineered VLP.

22. (canceled)

23. The composition of claim 21, wherein the naturally occurring plant virus or the engineered VLP binds and activates the TLR.

24. The composition of claim 21, wherein the naturally occurring plant virus or the engineered VLP comprises cowpea mosaic virus (CPMV), optionally wherein the CPMV is naturally occurring CPMV.

25. (canceled)

26. The composition of claim 21, comprising a plurality of the naturally occurring plant virus or the engineered VLPs, optionally wherein the plurality of the naturally occurring plant virus or the engineered VLPs are the same or different, and optionally wherein the plurality of the TLR agonists are the same or different

27-28. (canceled)

29. The composition of claim 26, wherein the plurality of the naturally occurring plant virus and the engineered VLPs comprise different TLR agonists.

30. The method of claim 7, wherein administration comprises injecting the CPMV into the tumor and subsequent cryoablation of the tumor.
